(19) United States
(12) Patent Application Publication
(10) Pub. No.: US 2016/0325109 A1
(43) Pub. Date: Nov. 10, 2016

(54) SYSTEM AND METHOD FOR MULTI-COLOUR LIGHT TREATMENT

(71) Applicant: MEDITECH INTERNATIONAL INC., Toronto (CA)

(72) Inventors: Alex Louis Knaus, Ontario (CA); Kaveh Seyed Momen, Ontario (CA)

(21) Appl. No.: 15/100,545

(22) PCT Filed: Jul. 17, 2014
(86) PCT No.: PCT/CA14/50680

§ 371 Date: May 31, 2016

Related U.S. Application Data
(60) Provisional application No. 61/847,303, filed on Jul. 17, 2013.

(51) Int. Cl. A61N 5/06

(52) U.S. Cl. CPC A61N 5/06; A61N 2005/0626; A61N 2005/0652; A61N 2005/0663; A61N 2005/0659

(57) ABSTRACT

A system and method for multi-colour light treatment is provided. The system comprises: a treatment head for emitting light from a light source to a user. The treatment head comprises: a first light emitting element operable to emit light at a first wavelength; at least one other light emitting element operable to emit light at a second other wavelength. The system further comprises a switch coupled to said treatment head, the switch configured for alternately switching between the first and other light emitting element in dependence upon pre-defined treatment criteria.

### TECHNICAL FIELD

The following relates generally to apparatus and method for treatment with light.

### BACKGROUND

Light treatment of patients for various conditions is becoming well known. Light treatment of injuries such as sport injuries and sprains as well as chronic conditions such as arthritis, sciatica, and chronic slow healing wounds or sores, are all well known.

The principle of all these light treatments is the application of light radiating in the area of the patient's condition. It is found that in order to be effective, the light source should be in close contact with the skin. The light source is usually an array or panel of light emitting diodes, or in some cases low level laser. The treatment typically becomes more effective over longer periods. The light sources may, for example, be left in contact with the skin for up to sixty minutes or more. This enables deep penetration of the light rays into the tissues, and has been found to be efficacious in many instances.

Various treatment protocols have been developed, some of which require different levels of radiation at varying intervals or the use of different wavelengths of radiation. Various wavelengths of radiation may be referred to as “colours”, irrespective of whether the wavelength is within the visible spectrum. The area to be treated may also extend beyond the treatment head, requiring repositioning of the treatment head during treatment.

The use of two or more colours of light sources introduces various disadvantages. For example, to provide light therapy at two colours, two separate treatment heads may be used, however, this complicates the task required of the operator of the treatment system, who must manually re-position the treatment heads onto the area being treated and does not allow for fast switching between wavelengths, or continual application of radiation.

Another option is to alternate between two or more colours of light sources in the same treatment head. For example, US publication number 2008/0065056 discloses a phototherapy device that may include multi-color LEDs for emitting at multiple wavelengths.

However, a disadvantage of multi-colour LED treatment heads is that LEDs of various colours will draw different levels of current when driven at the same voltage. This causes an increase in heat generated by the LEDs through which a higher current is drawn, which may be uncomfortable or even dangerous for the patient.

Furthermore, to achieve adequate and uniform illumination with a light treatment head, light sources within the head must be closely spaced. For example, a commonly used light source is a light emitting diode (LED) array. To deliver light at a sufficient intensity using an LED array light source, individual LEDs must be closely spaced in the array, which is more difficult to achieve with a multi-colour LED array since at any one time, only a fraction of the total number of LEDs on the treatment head are illuminated. The close spacing of the LEDs also exacerbates the heat dissipation problems mentioned above.

### Object of the Invention

It is therefore an object of the present invention to obviate or mitigate the above disadvantages.

### DISCLOSURE OF THE INVENTION

An embodiment of the invention will now be described by way of example only with reference to the accompanying drawings.

### SUMMARY OF THE INVENTION

In one aspect of the present invention, there is provided a system configured for multi-colour light treatment, the system comprises a treatment head. The treatment head comprises a first light emitting element operable to emit light at a first wavelength; at least one other light emitting element operable to emit light at a second other wavelength. The system further comprises a switch coupled to said treatment head, the switch configured for alternately switching between the first and other light emitting element in dependence upon pre-defined treatment criteria.

In another aspect, the system further comprises: a treatment head control module communicatively coupled to the treatment head, the treatment head control module comprising instructions for defining said pre-defined treatment criteria, the treatment head control module for providing at least one of: an intensity of emitted light; a duration of light emission at a particular wavelength; number of cycles of treatment at each wavelength; and a selection of at least two wavelengths to the treatment head for controlling operation of the light emitting elements.

In yet another aspect, the system comprises: a treatment database coupled to the treatment head control module, the treatment database comprising pre-defined treatment protocols associated with at least one patient, the pre-defined treatment protocols for further defining said pre-defined treatment criteria.

In yet another aspect, the system comprises a user interface communicatively coupled to the treatment head control module, the treatment head control module configured for receiving input from the user interface for further defining said pre-defined treatment criteria.

In yet another aspect, the switch further comprises a polarity switch operable to toggle between a forward and a reverse polarity to cause the treatment head to alternately emit light at the first wavelength by the first light emitting element and at the second wavelength of the other light emitting element.

In yet another aspect, the forward polarity is driven with a forward current and the reverse polarity is driven with a reverse current.

In yet another aspect, the system further comprises a current controller coupled to said first light emitting element, and said other light emitting element, the current controller for setting a pre-defined current value for driving a respective one of said first and said other light emitting element, the pre-defined current value associated with a characteristic of said light emitting element.

In yet another aspect, the current value set by the current controller is further associated with a pre-defined light intensity associated with the emitted light.

In yet another aspect, the system further comprises a dynamic voltage controller and wherein a selected one of the first and the other light emitting element is configured to emit light in dependence upon receiving, from said dynamic voltage controller, a forwardly biased input driving voltage above a pre-defined characteristic threshold voltage of the selected one light emitting element.

In yet another aspect, the voltage controller automatically adjusts the driving voltage to cause a constant current to be driven through the selected light emitting element at a selected current value associated with the respective light emitting element.

In yet another aspect, once the switch switches from the first to the other light emitting element, the voltage controller is configured to dynamically adjust the driving voltage to a pre-defined voltage value associated with the other light emitting element.

In yet another aspect, the current controller is further configured to receive an input from at least one of a user interface and a pre-defined treatment protocol stored on a memory for instructing the switch to activate based on said input.

In yet another aspect, the treatment head further comprises a temperature sensor for sensing a temperature associated with the treatment head and a current limiter configured for limiting electrical current flowing through said light emitting elements in dependence upon said sensed temperature being upon a pre-defined temperature value associated with the light emitting elements.

In yet another aspect, the current limiter comprises a positive temperature coefficient thermistor placed in series connection with the light emitting elements.

In yet another aspect there is provided a power control module comprising the switch, a current controller coupled to the switch and the light emitting elements for adjusting a current applied to said first and other light emitting elements, and a dynamic voltage controller for applying a driving voltage to each said first and other light emitting elements.

In yet another aspect, the power control module is positioned on the treatment head and electrically coupled thereto.

In yet another aspect, there is provided a method for multi-colour light treatment, comprising: applying a forward driving voltage to a bi-colour LED to cause the LED to emit at a first wavelength, the LED comprising a forward voltage upper threshold and a reverse voltage upper threshold; orienting the bi-colour LED to emit onto a treatment area of a patient; monitoring the voltage applied to the LED; and upon the voltage exceeding the forward upper voltage threshold, reducing the driving voltage of the LED.

### BRIEF DESCRIPTION OF THE DRAWINGS

FIG. 1A is a perspective view of an arm receiving light treatment above the elbow joint from a light treatment system in accordance with one embodiment;

FIG. 1B is a perspective view of an arm receiving light treatment below the elbow joint from a light treatment system in accordance with one embodiment;

FIG. 2 is a block diagram of a system for administering light treatment in accordance with one embodiment;

FIG. 3A is a top perspective view of an example LED treatment head in accordance with one embodiment;

FIG. 3B is a bottom view showing a light source for the example LED treatment head of FIG. 3A in accordance with one embodiment;

FIG. 4 is a block diagram of a power control module of a bi-colour LED array in accordance with one embodiment;

FIG. 5 is a block diagram of an example multi-colour LED treatment head in accordance with one embodiment;

FIG. 6A is a simplified circuit diagram illustrating a switch for switching the polarity of a bi-colour LED array in accordance with one embodiment;

FIG. 6B is an enlarged view of a simplified circuit diagram of a bi-colour LED array in accordance with one embodiment;

FIG. 6C is a simplified circuit diagram of FIG. 6A shown with the switch in a first position in accordance with one embodiment;

FIG. 6D is an enlarged view of a simplified circuit diagram of a bi-colour LED array biased in a first direction in accordance with one embodiment;

FIG. 6E is a simplified circuit diagram of FIG. 6A shown with the switch in a second position in accordance with one embodiment;

FIG. 6F is an enlarged view of a simplified circuit diagram of a bi-colour LED array biased in a second direction in accordance with one embodiment;

FIG. 7 is a process flow diagram of an example process for switching between colours in a bi-colour treatment head in accordance with one embodiment;

FIGS. 8A through 8E constitute a process flow diagram of an example process for operating a bi-colour LED treatment head in accordance with one embodiment;

FIG. 9 is a screen capture of an example user interface for receiving input to enter patient information in accordance with one embodiment;

FIG. 10 is a screen capture of an example user interface to receive a selection for selecting a treatment from a treatment database in accordance with one embodiment;

FIG. 11 is a screen capture of an example user interface for entering prescription information for a single step treatment in accordance with one embodiment;

FIG. 12 is a screen capture of an example user interface for entering prescription information for the first step of a two-step treatment using a first colour of light source;

FIG. 13 is a screen capture of an example user interface for entering prescription information for the second step of a two-step treatment using a second colour of light source in accordance with one embodiment;

FIG. 14 is a screen capture of an example user interface for displaying treatment information to an operator in accordance with one embodiment;

FIG. 15 is a screen capture of an example user interface for confirming start of treatment in accordance with one embodiment;

FIG. 16 is a screen capture of an example user interface for displaying treatment information to an operator during operation of the light treatment system in accordance with one embodiment;

FIG. 17 is a screen capture of an example user interface for notifying an operator that a treatment is complete in accordance with one embodiment;

FIG. 18 is a screen capture of an example user interface for entering prescription information into the treatment head controller for two-step treatment using a modulated light source in accordance with one embodiment; and

FIG. 19 is a screen capture of an example user interface for creating a new treatment for use by the LED treatment head in accordance with one embodiment.

### DETAILED DESCRIPTION OF THE DRAWINGS

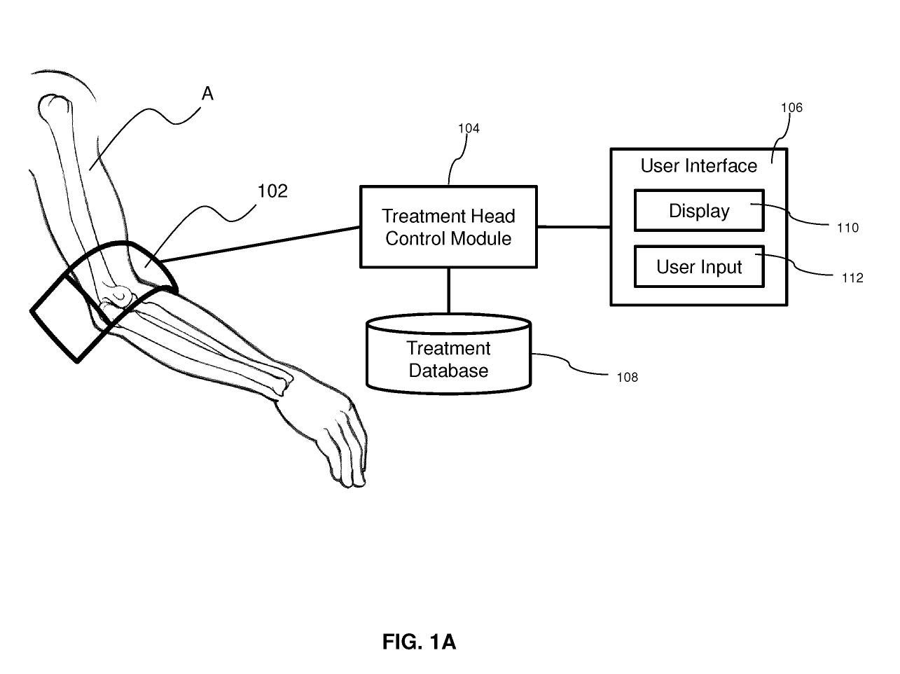

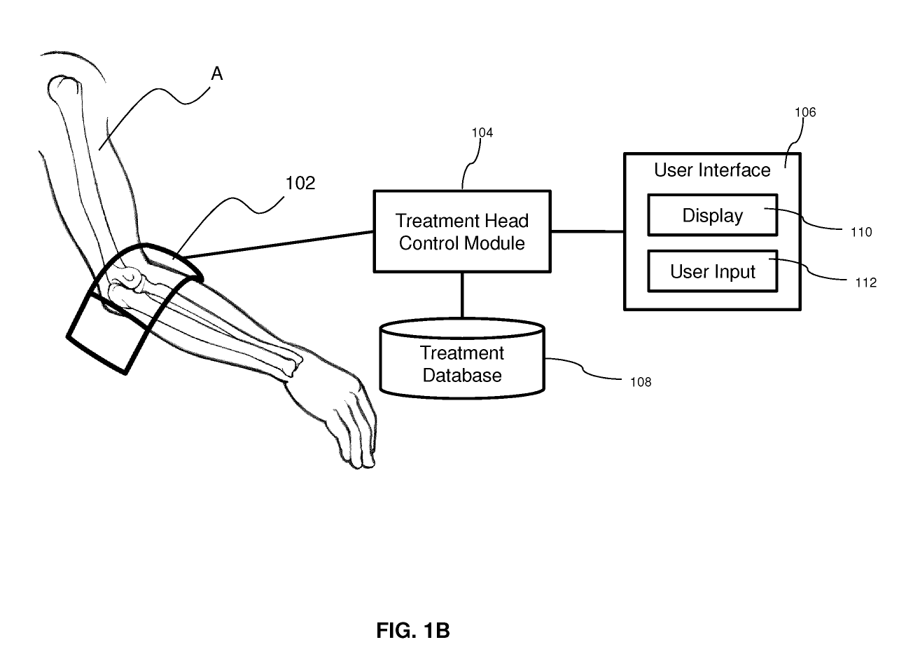

An example of a light treatment system 100 is provided in FIGS. 1 through 3. The light treatment system 100 comprises a treatment head 102, a treatment head control module 104, a user interface 106, and a treatment database 108. The user interface 106 further comprises a display 110 and a user input interface 112.

The treatment head 102 can be oriented and located onto the skin of patient and is operable to emit light from a light source 103 onto a desired area of the patient at two or more wavelengths. An example of such a treatment head is shown in U.S. patent application Ser. No. 13/355,162, the contents of which are incorporated herein by reference.

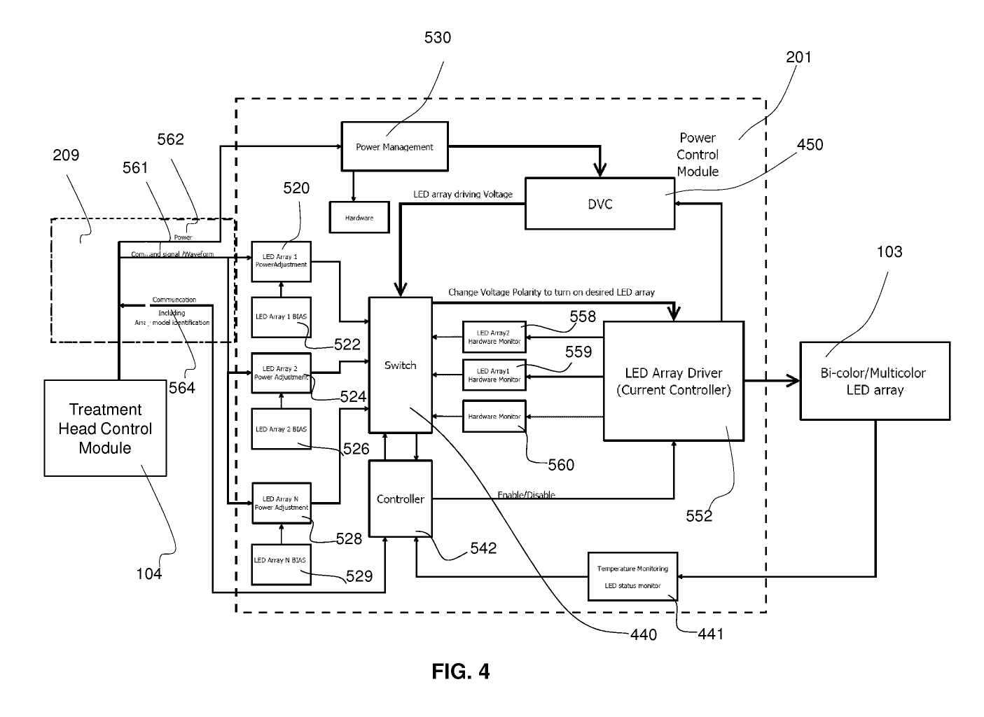

The treatment head 102 comprises a power control module 201 which controls power for delivery to the light source 103. An example bi-colour treatment head power control module 201 is shown in greater detail in FIG. 4.

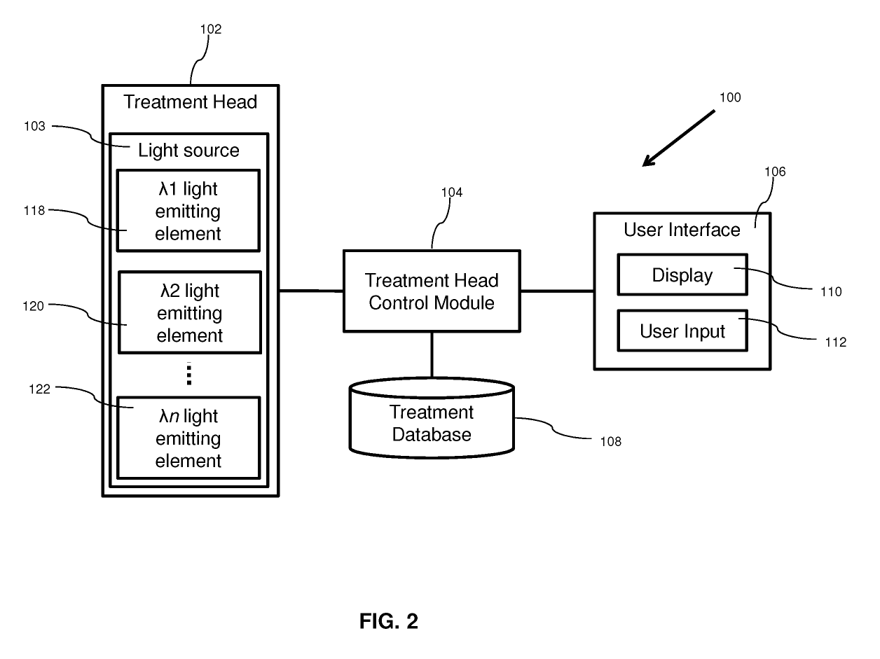

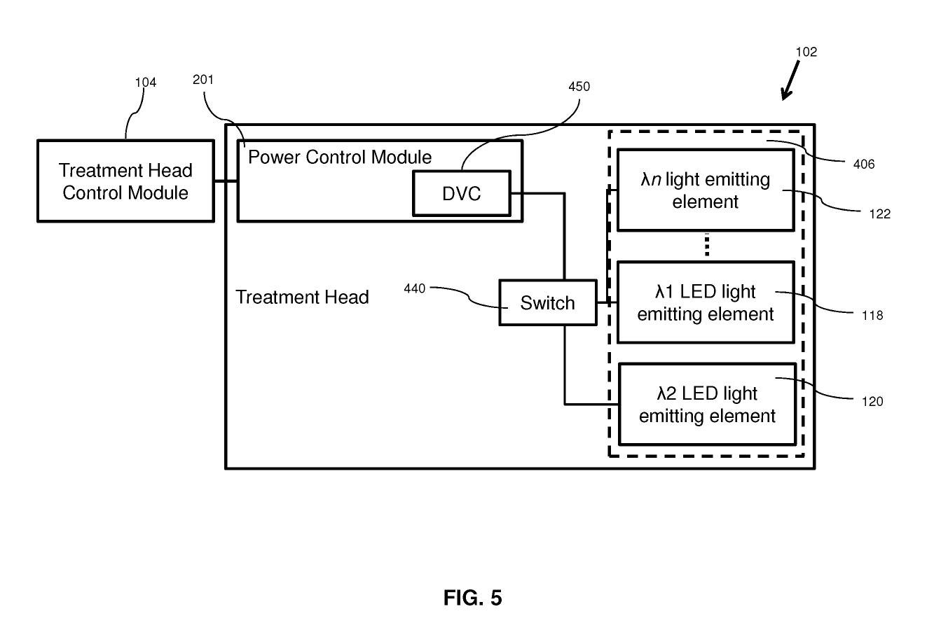

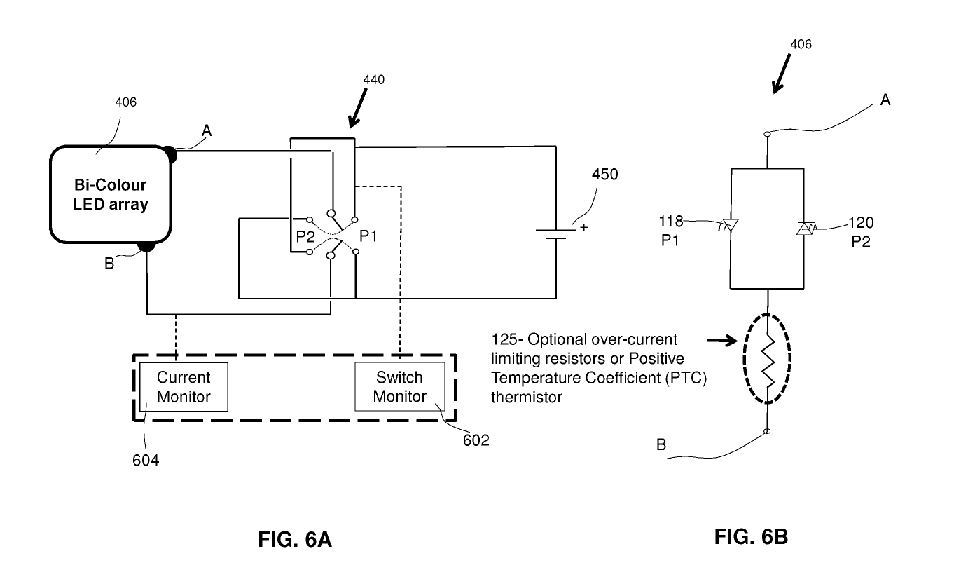

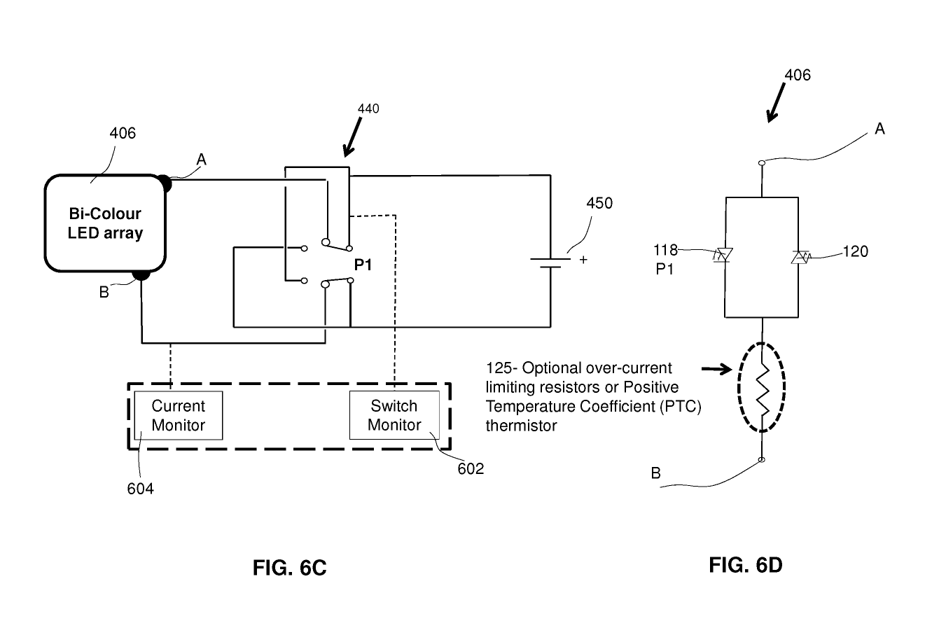

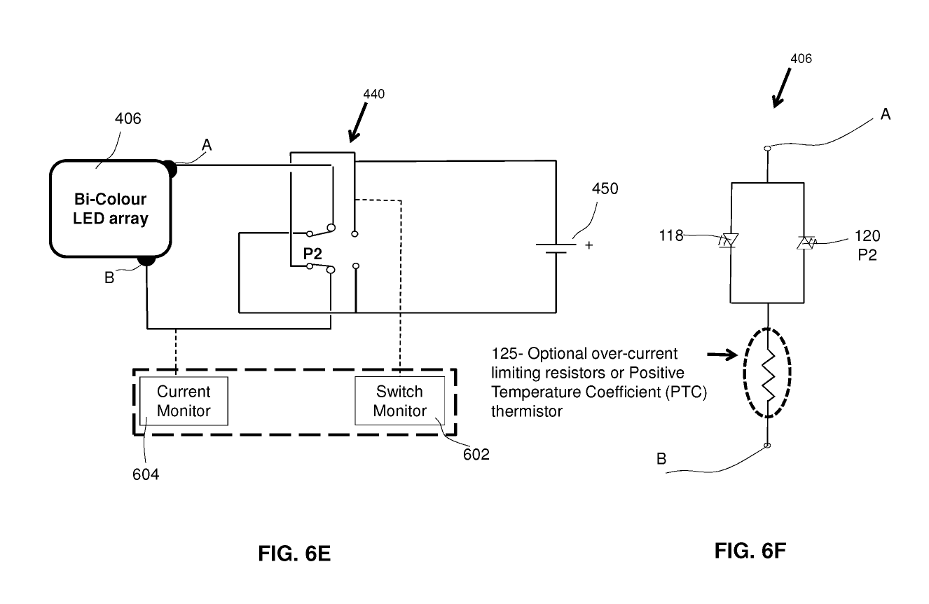

Referring to FIGS. 2-4, to emit at two or more wavelengths, the light source 103 comprises at least one light emitting element 118 operable to emit at a first wavelength and at least one other light emitting element 120 operable to emit at a second and distinct wavelength. The power control module 201 comprises a switch 440, as shown in FIG. 4. The switch 440 is configured to selectively cause the light emitting elements 118 and 120 to emit (e.g. as shown in FIG. 4 and FIG. 5), depending upon the switch positioning and parameters. For example, the switch 440 may enable neither light emitting element 118, 120 to emit, both light emitting elements 118, 120 to emit simultaneously, or to select between the light emitting elements 118 and 120. An example of a switch having three positions is the polarity switch provided below with reference to FIGS. 6A through 6E. As will be further described herein, the polarity switch enables the first light emitting element 118 to emit in a first position, the second light emitting element 120 to emit in a second position and neither of the light emitting elements to emit when the switch is in a third position.

As will be appreciated, the light source 103 may further comprise additional light emitting elements (e.g. light emitting element 122) which are switchable by switch 440 and enable the treatment head 102 to emit at additional, or alternate, wavelengths. For example, the light source 103 may comprise three, four, five, or more light emitting elements. It will also be appreciated that one or more of the light emitting elements may emit at a plurality of wavelengths, for example, a light emitting element may emit substantially white light.

The treatment head control module 104 controls the operation of the treatment head 102 and is in communication with the treatment head 102, the user interface 106, and the treatment database 108. The treatment head control module 104 comprises a memory for storing computer executable instructions and a processor for executing the computer executable instructions stored in the memory.

The treatment head control module 104 controls parameters associated with the light source 103 of the treatment head 102. Specifically, the treatment head control module 104 is operable to control various parameters of the light source 103 including, for example, the intensity of emitted light, the duration of light emission, the number of cycles of treatment to be applied to a particular area of the patient, and the wavelength of light emitted onto the patient. Such parameters are further controlled by communication between the power control module 201 (e.g. shown in FIG. 4) and the treatment head control module 104 for defining and adjusting one or more of the following parameters: the switch 440 positioning; the voltage polarity for turning on a selected LED array (and its light emitting elements); a pre-defined threshold value for each LED array driving voltage (and its light emitting elements); and a pre-defined threshold value for the current driven through each LED array (and its light emitting elements).

The treatment head control module 104 further comprises, or is communicatively and/or electrically linked to, a power source which powers the light source 103 (e.g. power control module 201 in FIG. 4). In the example embodiment provided with reference to FIG. 4, the power source is incorporated into the treatment head control module 104. The treatment head 102 may otherwise, or in addition, include an on-board power source such as a battery to power the light source 103.

As outlined above, the treatment head control module 104 is in communication with the user interface 106. The user interface 106 is configured to obtain instructions from an operator of the light treatment system 100 via a user input 112 and provide information to an operator via an output, for example, a display 110. The user interface 106 may otherwise, or in addition, include a speaker, one or more indicator lights, a microphone, or various other input and output devices known in the art.

In one embodiment, the user interface 106 enables a user to provide an input to control the operation of the light source 103 on the treatment head 102 and to receive data from the treatment head 102 via the treatment head control module 104. This data may include, for example, the operational status of the treatment head 102 (i.e. to determine whether the treatment head is in an operating condition), the operational parameters of the treatment head 102 (i.e. the wavelength(s) and waveform at which the light source 103 is emitting), the temperature of the treatment head 102, and other information relevant to an operator of the light treatment system 100.

The treatment head control module 104 is in communication with a treatment database 108, which is operable to store various treatment protocols. The treatment database 108 may also store patient information including a patient identifier, patient health information, treatment history, injury diagnosis, prescriptions, and other relevant information. Treatment protocols can be selected by an operator based on a patient's prescription of a pre-existing treatment protocol or based on a customized treatment protocol. A treatment protocol includes a combination of treatment steps performed in a treatment session with a patient. An operator of the light treatment system 100 may select an appropriate treatment protocol from the treatment database 108 via the user input 112 of the user interface 106 and view treatment instructions, progress, and other information via the display 110 of the user interface 106. The treatment head control module 104 may select a treatment protocol from the treatment database 108 based on patient identifier stored in a patient database (not shown) and linked to a treatment protocol.

New treatment protocols may be entered into and stored in the treatment database 108 via the user interface 106. Existing treatment protocols may also be linked with a patient identifier via the user interface 106. In one example, the treatment head control module 104 is operable to obtain additional treatment protocols, for example, via a network connection (not shown) and store these in the treatment database 108. These additional treatment protocols may then be linked with selected patient identifiers or selected by an operator of the light treatment system 100 for use during a treatment session.

A primary function of the treatment head 102 is to emit light onto the area of the patient being treated. It will be appreciated that various geometries and styles of treatment heads 102 may be used depending on the specific application. By way of example only, an elongate, substantially planar treatment head may be used to treat the entire spine of a patient whereas a shorter, highly flexible treatment head may be used to treat a patient's arm, knuckles or wrist. It will be appreciated that any treatment head 102 comprising a light source 103 which can illuminate the area of a patient being treated to provide the desired therapeutic effect may be used.

The diagrams of FIGS. 1A and 1B show exemplary applications of the treatment head 102. In FIG. 1A, a treatment head 102 can be located above an elbow joint of a patient's arm A to treat the area above the joint, whereas the treatment head 102 is located below the elbow joint of the patient's arm A in FIG. 1B to treat the area below the joint. Light treatment may be used to treat the elbow joint itself, treat tendonitis in the triceps, or treat osteoarthritis in the joint itself, and the location of the head is selected to promote adequate exposure of the area being treated. It will be appreciated that the treatment head 102 may be used to treat various other conditions and various other areas of a patient's body.

A treatment head 102 comprising a light source 103 is shown in FIG. 2. In one embodiment, the power control module 201 is part of the treatment head 102 and in another embodiment; the power control module 201 is communicatively and/or electrically coupled to the treatment head 102). The light source 103 comprises light emitting elements 118 and 120 which emit at a first wavelength and a second other wavelength, respectively. The light source 103 may further include additional light emitting elements 122 that emits at various other wavelengths.

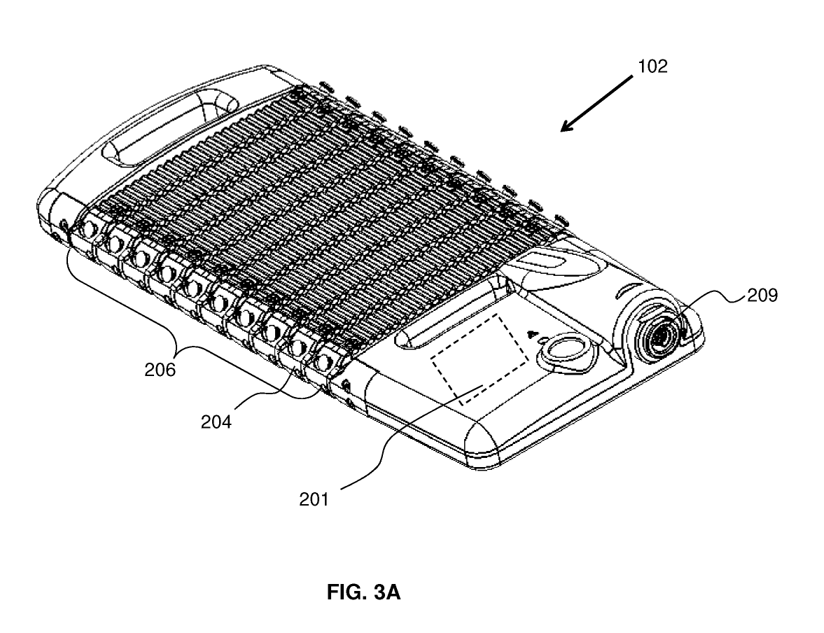

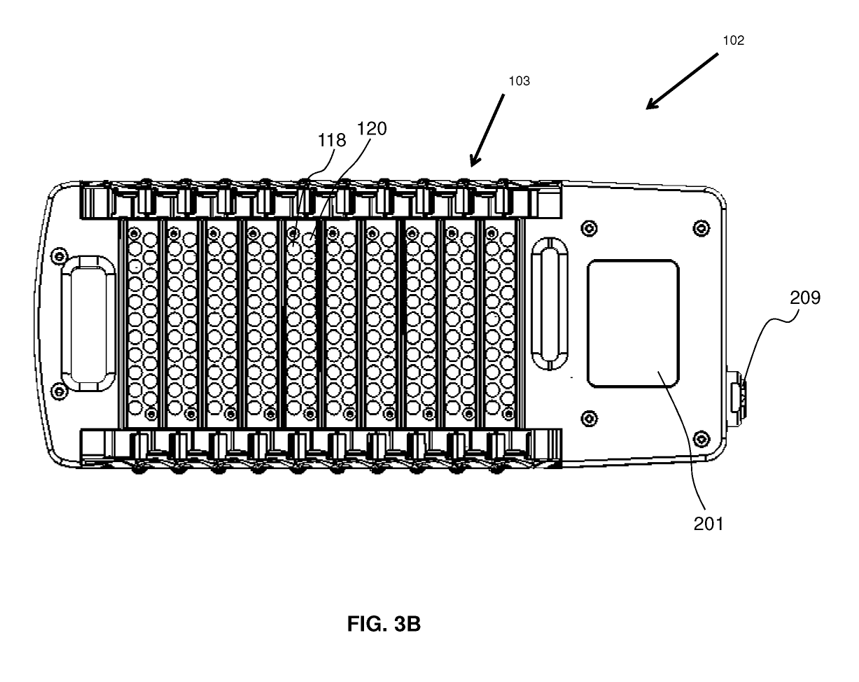

Referring to FIGS. 3A and 3B, a power control module 201 is provided in an exemplary light treatment head 102. In the example of FIGS. 3A and 3B, the power control module is located within a housing of the light treatment head 102 and is not directly visible. A light source 103 is located on the underside of the light treatment head 102 and is preferably supported by individual rigid portions 204 interconnected by a hinge apparatus 206 to contour a patient's body for close contact between the light source 103 and the patient's skin. It will be appreciated that the light source 103 may otherwise comprise a continuously flexible light source 103 or a completely rigid light source 103 in various configurations adapted to provide light treatment to a patient. It will also be appreciated that other shape configurations and/or flexibility variations of the light treatment head 102 may be envisaged for providing light treatment.

The treatment head 102 comprises a port 209 having a power interface and a data communication interface for receiving power from a power source and for communicating with the treatment head control module 104, respectively. It will be appreciated that the power interface may be separate from the communication interface. It will also be appreciated that the treatment head 102 may be in wireless communication with the treatment head controller. The treatment head 102 may otherwise, or in addition, receive power wirelessly via a wireless power transmission protocol, which may include, by way of example only, the Qi™ interface standard or the WiPower™ interface standard.

The light source 103 comprises one or more light emitting elements 118, 120 disposed along the treatment surface of the light treatment head 102 (see FIG. 3B). The light emitting elements 118, 120 may comprise LEDs including organic LEDs (OLEDs), fibre optics coupled to a light guide, LASER emitters, or various other light emitting structures and combinations thereof known in the art. It will be appreciated that any light emitting element which provides the energy and intensity of light to achieve the desired therapeutic effect may be used. It will also be appreciated that the light emitting elements 118, 120 may be chosen based on additional properties including their heat generating characteristics of the light source 103, the physical size of the light emitting elements 118, 120, the spectral width of a light emitting element, or the electrical efficiency of a light emitting element 118, 120, and based on other design considerations that would be apparent to a person familiar with light treatment systems.

In one aspect, the light source 103 further directly comprises the temperature and LED status monitoring 441 for monitoring the temperature and status operation of one or more of the light emitting elements 118, 120, and 122. The operation of the temperature and LED status monitoring 441 has further been defined with reference to FIG. 4.

In one embodiment, the light source 103 comprises safety features such an over-current and over-temperature protection mechanisms. In one aspect, the light source 103 comprises a Positive Temperature Coefficient (PTC) thermistor 125 as an example of a component that limits the electrical current flowing through the light emitting elements (e.g. 118, 120, 122). Accordingly, by controlling the electrical current flowing through the light emitting elements 118, 120, 122, the light source 103 temperature is also controlled as the more current that flows through the light emitting elements, the higher the temperature of the light source 103. Referring to FIGS. 6A to 6F, the PTC thermistor 125 can be placed in series with the light emitting elements 118, 120 at either point A or B or both. In normal operation, the PTC thermistor 125 acts like a resistor. In a fault event, where higher uncontrolled current can flow through the light emitting elements 118, 120 (or strings of these elements), the PTC thermistor 125 is configured to dynamically increase its resistance to control the current flowing through the light emitting elements. If the excess current is higher than the PTC trip current (e.g. a pre-defined current threshold) then the PTC is configured to function as a fuse to cut the connection between the bi-colour LED array 406 and the dynamic voltage controller (DVC) 450 at point A or B or both, depending on where PTC thermistor 125 is connected. Other current controlling components can be envisaged for controlling the electrical current flowing through the light emitting elements such as to result in controlling the respective temperature of the light source 103 and maintaining it below a pre-defined threshold.

In use, the light emitting elements 118, 120 are placed in contact with, or in close proximity to, a patient's skin in the area being treated. This placement maximizes penetration of the light emitted by the light source 103 and improves distribution of the light into the area of the patient being treated to maximize the therapeutic effect of the treatment.

Typically, the wavelength and intensity of light emitted by such light emitting elements 118, 120 are relevant to the therapeutic effect. The light emitting elements 118, 120 may emit in the visible range, in the near-infrared, or in the infrared range. The light emitting elements 118, 119 may otherwise, or in addition, emit in the ultra-violet range, for example, to sanitize a portion of a patient's skin, for example, in the vicinity of a wound. By way of example only, wavelengths of light emitting elements that may be used in the treatment head 103 include 450 nm, 660 nm, 830 nm, 840 nm, and 905 nm. It will be appreciated that various other wavelengths may be used.

Combinations of light emitting elements 118, 120 are used to create a multi-colour treatment head. For example, a treatment head 102 can comprise an array of LEDs, a first group of which comprise light emitting element 118 and emit at a first wavelength 660 nm while a second group comprising light emitting element 120 and emit at a second wavelength 840 nm. The LEDs may, for example, be arrayed such that the LEDs of the first light emitting element 118 are substantially evenly distributed throughout the treatment head and the LEDs of the second light emitting element 120 are also substantially evenly distributed throughout the treatment head.

The light emitting elements 118, 120 may also emit at various output powers. For example, the output power of the light source 103 may be 750 mW or 1500 mW.

As mentioned above, traditional bi-colour or multi-colour light sources can contribute to excess heat generation and are difficult to pack tightly to provide a sufficiently high intensity light source. It has been realized that these factors can be at least somewhat mitigated by using a bi-colour LED that can alternate emission between a first wavelength and a second wavelength depending on the polarity in which the LED is being driven. The use of bi-colour or multi-colour LEDs also provides a light source 103 which is operable to emit two or more colours of light from the same LED package, thereby potentially increasing the density of an LED array.

In the specific example embodiment of a bi-colour LED array, the light emitting elements 118, 120 comprise bi-colour LED modules which emit at a first wavelength when forwardly driven but emit at a second, distinct wavelength when the polarity of its terminals is inverted. Such bi-colour LED modules are effectively two separate LEDs which are manufactured on the same silicon substrate such that the active region of each of the two LEDs is adjacent and can be located within the same LED package. The anode of the first LED is in electrical communication with the cathode of the second LED at a first electrical terminal, and similarly, the cathode of the first LED is in electrical communication with the anode of the second LED at a second electrical terminal. A current will flow across an LED if the LED is forwardly biased at a level higher than its characteristic threshold voltage, but will not emit light if it is reverse biased or if it is forwardly biased but below the characteristic threshold.

Therefore, when a driving voltage above a characteristic threshold value is applied to the bi-colour LED such that the first terminal is at a higher voltage than the second terminal, the first LED emits whereas the second LED does not. Conversely, when a driving voltage above a characteristic threshold is applied to the bi-colour LED such that the first terminal is at a lower voltage than the second terminal, the second LED emits whereas the first LED does not. In this way, the bi-colour LED array is configured to receive input for alternately toggling the operation of the first and the second LEDs, thereby alternately emitting at one of a first and a second wavelength associated with a respective one of the first and the second LED of the bi-colour LED array. In accordance with one embodiment, the selection of the first and second LED is thus dependent upon which light emitting element is forwardly biased at a given time and provided with a pre-defined driving voltage higher than the characteristic voltage for the respective first and second LED (e.g. light emitting elements 118 and 120). The driving voltage provided to the respective light emitting element 118, 120 can be controlled by the dynamic voltage controller (DVC) 450 shown in FIG. 4.

Since the bi-colour LED can be provided in a single package, the size of such a bi-colour LED may be similar to the size of an equivalent monochrome LED, enabling greater LED densities in an LED array. An embodiment of a treatment head comprising a bi-colour reversible polarity LED is provided below with reference to FIGS. 5 and 6.

By way of example, a 660 nm light array at 750 mW total output power can be provided using approximately 180 LEDs and interspersed with another array of 180 840 nm LEDs at 750 mW total output power to provide 1500 mW of total output power. Other output intensities are possible. Additionally, light emitting elements emitting light at other wavelengths could be provided, in particular to provide different types of treatment.

Turning now to FIG. 4, a simplified block diagram of a power control module 201 for controlling the operation of a bi-colour LED treatment head 102 is provided. As discussed with reference to FIG. 4, the power control module 201 is configured to control and modify parameters such as, but not limited to: driving voltage, current flowing through LEDs, selection of one or more LEDs, control of temperature, consistency of current flowing through LEDs, and switch position selection. In one aspect, the power control module 201 is further configured to control such parameters depending on user input from the user interface 106 and/or pre-defined threshold values as provided by the treatment head control module 104 and/or stored on the power control module 201.

The treatment head control module 104 is shown in communication with the power control module 201 on the treatment head 102. The power control module 201 is operable to provide power to, and control, one or more light emitting elements (e.g. 118, 120, 122), or arrays of light emitting elements. The power control module 201 may drive a first light emitting element operable to emit at a first wavelength 118, a second light emitting element operable to emit at a second wavelength 120. The power control module 201 may further be operable to drive up to n additional light emitting elements 122.

The power control module 201 comprises a port 209 with a power interface 562 to provide power to a power management module 530, and a communication interface 561. The power management module 530 comprises a DC to DC converter. The power management module 530 may alternately, or in addition, comprise an AC to DC converter, or an equivalent circuit to increase or decrease power to a selected level for one or more light emitting elements for receiving AC power.

The power management module 530 is operable to power a dynamic voltage controller (DVC) 450, which is configured to apply a driving voltage to first, second, and n^{th} LED light emitting elements 118, 120, and 122 in the light source 103 via the switch 440. In one aspect, the dynamic voltage controller 450 cooperates with the switch 440 for triggering on one of the light emitting elements 118, 120 in dependence upon the value of the driving voltage being greater than the characteristic voltage and based upon the switch polarity as described herein.

The interface 209 also includes a communication connection 564, which enables the treatment head control module 104 to communicate with a controller 542 on the power control module 201. The controller 542 may comprise a microcontroller, FPGA, or other processing circuit and is linked to the switch 440 and operable to actuate the switch 440.

For example, in a bi-colour light source 103 comprising only a first light emitting element 118 and a second light emitting element 120, the controller 542 is operable to actuate the switch 440 to selectively cause the first and second light emitting elements 118 and 120 to emit.

In one example, the switch 440 is a polarity switch which is operable to toggle between a forward and a reverse polarity and the first and second light emitting elements 118 and 120 comprise a bi-colour LED. As such, when the switch 440 is actuated to cause the bi-colour LED to alternate between a forward and a reverse polarity, the colour of emission from the bi-colour LED light source 103 is switched from a first wavelength emitted by the first light emitting element 118 to a second wavelength emitted by the second light emitting element 120.

Specifically, the first LED light emitting element 118 is operable to emit light at a first wavelength when driven with a forward current whereas the second LED light emitting element 120 is operable to emit light at a second wavelength when driven with a reverse current. The first LED light emitting element 118 may be combined with the second LED light emitting element 120 to form a common bi-colour LED array. Such an array enables the light emission from the first LED light emitting element 118 and second LED light emitting element 120 to be substantially uniformly distributed across the surface of the bi-colour LED light source 103.

Bias circuits 522 and 526 are provided to bias the first and a second LED light emitting elements 118 and 120 in their respective operational regimes. Additional bias circuits 529 may be provided to bias n additional light emitting elements 122. Power adjustment modules 520, 524, and 528 associated with each of the light emitting elements 118, 120, and 122, respectively, are operable to receive an input waveform via the command signal 561 from the treatment head control module 104 and provide the input waveform to the polarity switch 440 to cause each colour of bi-colour LED array to be emitted in accordance with the input waveform. For example, the power adjustment modules 520, 524, 528 may enable respective ones of the bi-colour LED array to emit in a modulated, sinusoidal waveform and at a selected duty cycle.

Each of the light emitting elements 118, 120, and 122 is in communication with a current controller 552. The current controller 552 serves as a current source or a current sink (depending on whether there is a need to increase or decrease the current). The current controller 552 is operable to control and/or adjust the current applied to the light emitting elements 118, 120, and 122 based on an input from the treatment head control module 104 and/or depending on pre-defined current thresholds and/or pre-defined light intensity thresholds (related to the current value) associated with the light emitting elements 118, 120. For example, the current may be selected depending on the current requirements of a particular light emitting element.

The current controller 552 interfaces with LED hardware monitors 558, 559, and 560 associated with each of the LED light emitting elements 118, 120, 122, respectively. The LED hardware monitors 558, 559, and 560 monitor the current flow through arrays of the LED light emitting elements 118, 120, and 122, respectively. For a given intensity of optical output, the current controller 552 maintains the current being driven through each of the LED light emitting elements 118, 120, and 122 substantially constant at a selected current. The current at which an LED light emitting element is driven depends on the power requirements of the LED light emitting element and the desired optical output.

The DVC 450 is operable to dynamically adjust the voltage applied to the LED light emitting elements 118, 120, 122 based on a reading of a voltage in the current controller 552. For example, the DVC 450 may receive feedback from a voltage splitter on the current controller 552 and dynamically adjust the voltage being applied such that the applied voltage is higher than a lower voltage threshold and lower than an upper voltage threshold. The lower voltage threshold is selected to be at, or above, the voltage level required to drive the LED light emitting elements 118, 120, and 122. Maintaining the voltage above a lower voltage threshold enables the LED light emitting elements 118, 120, 122 to emit. Maintaining the voltage below an upper threshold prevents the DVC 450 from applying an unnecessarily high driving voltage, which can lead to excess heat generation within the driving circuitry. The current controller 552 is also configured to prevent an excess voltage from being applied by the DVC 450 by providing a voltage reading to the DVC 450, which enables the DVC 450 to dynamically adjust the driving voltage such that the driving voltage falls within a selected range.

Example driving voltages of LED light emitting elements may range from about 1 volt up to 3.5 volts or more. For example, an LED light emitting element which emits at 660 nm may require 2.1 to 2.3 volts whereas an LED light emitting element which emits at 840 nm may require only 1.5 to 1.7 volts.

Upon the DVC 450 detecting a voltage, via the current controller 552, that is above a selected threshold, the DVC 450 reduces the driving voltage being applied to the light source 103.

An LED status monitor 441 monitors other parameters of the LED light emitting elements such as temperature, or whether one of the LED light emitting elements is experiencing an electrical fault such as a short. The current controller 552 obtains the current, and any other available parameters, from the hardware monitors 558, 559, 560, and 441 and may adjust the current accordingly.

As mentioned above, the voltage required to drive an LED light emitting element in an LED array depends on parameters associated with that LED array. Specifically, power requirements of an LED light emitting element that emits at a first wavelength may be different from the power requirements of an LED light emitting element that emits at a second wavelength. For example, an LED array emitting at 660 nm may require a 22 volt applied voltage to achieve a particular current whereas an LED array emitting at 830 nm may require a 15 volt applied voltage to achieve the same current. As such, if the applied voltage is not reduced from approximately 22 volts to approximately 15 volts when switching a bi-colour LED light source 103 from a 660 nm LED array to the 830 nm LED array, although the LED array current is kept constant, the excess voltage creates significant heat in the drive circuit, which may eventually be a discomfort or even burn hazard to the patient and may also damage the light treatment system 100. Therefore, when the light source 103 is switched by switch 440 from a higher voltage LED array to a lower voltage LED array, the DVC 450 detects that the voltage across the drive circuit is above a selected threshold and the DVC 450 reduces the driving voltage of the array to reduce the waste heat produced by the drive circuit.

As such, the simplified circuit schematic provided in FIG. 4 compensates for the voltage requirements of various LEDs when switching between emission colours of a bi-colour LED array (e.g. switching from a first light emitting element 118 to a second light emitting element 12) or a multi-colour LED array to maintain the current below a selected threshold or within a selected range. For example, to generate approximately 1000 mW of optical output at 660 nm, a current of approximately 180 mA must be applied to the array of red LED light emitting elements. To generate approximately 2000 mW of optical output at 840 nm, a current of approximately 400 mA must be applied to the array of infrared LED light emitting elements.

Although the power control module 201 of FIG. 4 is explained in the context of using a bi-colour LED array as a light source 103, it will be appreciated that multi-colour arrays may also be used. For example, the LED array may comprise three or more colours, in which case, the switch is operable to select between the three LED colours, or between combinations of these three colours. Upon the DVC 450 determining, via the current controller 552, that the voltage has exceeded a selected voltage threshold, the DVC 450 reduces the driving voltage, thereby maintaining the driving voltage below the selected threshold.

As will be appreciated, the voltage applied to an LED must be higher than a characteristic threshold voltage to cause an LED to emit. It will be appreciated that biasing circuits 522, 526, and 529 are used to maintain each of the LED light emitting elements 118, 120, and 122 above their respective threshold voltages (e.g. associated with the characteristic threshold voltage). For example, biasing circuits 522, 526, and 529 may apply a voltage to an LED that, when combined with a signal voltage, produces a driving voltage that is above the threshold voltage of the LED.

Accordingly, as described, upon switching from a first light emitting element 118 to a second light emitting element 120, the DVC 450 verifies whether the driving voltage is above a characteristic value associated with driving the second light emitting element 120 and below a pre-defined threshold value (e.g. associated with causing excess heat generation) and thus the DVC 450 adjusts accordingly.

The biasing circuits 522, 526, and 529 may, for example, be operable to produce a biasing voltage that is at or slightly higher than the threshold voltage of the LED. If the threshold voltage of a first LED of a bi-colour LED is 1.9 volts whereas the second LED of the bi-colour LED is 1.8 volts in the reverse polarity, biasing circuit 522 may apply a 1.9 volt bias in the forward direction when the switch 440 is in the forward position whereas biasing circuit 526 may apply a 1.8 volt bias in the reverse direction when the switch 440 is in the reverse position. As such, the voltage applied by the DVC 450 to drive the LED can be below the threshold voltage before being biased by the biasing circuits 522 and 526.

This bias enables the LED light emitting elements 118 and 120 to illuminate substantially immediately following the actuation of the switch 440 and ensures that the LEDs are driven with a voltage above the minimum threshold. Moreover, the bias assists the power control module 201 in linearly controlling the optical output of the LED light emitting elements 118 and 120 as the current is varied.

As is shown from the simplified diagram of FIG. 5, the power control module 201 may further drive additional light emitting elements 122, having n different wavelengths, respectively. It will be appreciated that two or more light emitting elements may also share the same wavelength. FIG. 5 is a simplified example of a multi-colour treatment head 102 operable to select among up to n light emitting elements 118, 120, and 122 emitting at λ₁, λ₂, and An, respectively. The DVC 450 located in the power control module supplies a voltage to each of the light emitting elements 118 120, and 122. Switch 440, located between the DVC 450 and the light emitting elements 118, 120, and 122 is operable to switch between each of the light emitting elements 118, 120, and 122.

Referring now to FIG. 6A, a simplified circuit diagram shows the construction of an exemplary polarity switch 440 for toggling the bi-colour LED array. The polarity switch 440 is located between a DC power source and the bi-colour LED array. The power source is preferably the DVC 450 as is shown in FIG. 5, but may alternatively comprise a battery, transformer, or another type of DC power source. For simplicity, the biasing circuits 522 and 526 are not shown, however, it will be appreciated that they may be integrated with the power source or separate from the power source.

Specifically, the polarity switch 440 has a first position P1 and a second position P2 and is operable to toggle between P1 and P2. The switch is shown in position P1 in FIG. 6C and in position P2 in FIG. 6E. The polarity switch 440 may also have a third, neutral position (not shown), which prevents a driving voltage from being applied to the bi-colour LED array 406.

As will be appreciated from FIGS. 6A and 6C, when the polarity switch is in position P1, a positive voltage is applied to terminal A while a negative voltage is applied to terminal B. Conversely, when the polarity switch is in position P2, a negative voltage is applied to terminal A, while a positive voltage is applied to terminal B.

A current monitor 604 is provided in the circuit to monitor the current flow through the bi-colour LED array 406. As is explained further below, readings from the current monitor 604 can be used to adjust the driving current and voltage of the bi-colour LED array 406 to bring the current below a selected current threshold or to a selected current range.

A switch monitor 602 may also be provided to determine the instantaneous condition of the polarity switch 440. The treatment head control module 104 may obtain a reading from the switch monitor 602 to determine the position of the switch and to indicate to the user via the user interface 106 the colour at which the bi-colour LED array 406 is emitting.

Referring now to FIG. 6B, a simplified schematic diagram of the bi-colour LED array comprising a first light emitting element 118, which emits at a first wavelength, and a second light emitting element 120, which emits at a second wavelength. As can be seen from FIG. 6B, the anode of the first light emitting element 118 and the cathode of the second light emitting element 120 are in communication with terminal A. Conversely, the cathode of the first light emitting element 118 and the anode of the second light emitting element 120 are in communication with terminal B.

Not shown in FIGS. 6A through 6F is the biasing circuits 522 and 526. It will be appreciated that the biasing circuits add a biasing voltage to the power source 450 to ensure that the bi-colour LED light emitting elements in the bi-colour LED array 406 are being driven above their threshold voltage. It will also be appreciated, biasing circuits 522 and 526 may comprise a single biasing circuit in communication with, or incorporated in the power source or DVC 450. For example, a single biasing circuit may be provided to ensure that the voltage provided by the DVC 450 remains above the threshold voltage of either of the LEDs in the bi-colour LED array 406.

Therefore, when a positive voltage above a threshold voltage of the first light emitting element 118 is applied to terminal A with respect to terminal B, the first light emitting element 118 is illuminated while the second light emitting element is not. Conversely, when a negative voltage above a threshold voltage of the second light emitting element 120 is applied to terminal A with respect to terminal B, the first light emitting element 118 is not illuminated while the second light emitting element is illuminated.

Referring again to FIGS. 6C and 6E, as well as 6D and 6F, it can be seen that when the polarity switch 440 is in position P1, the first light emitting element 118 is emitting while the second light emitting element 120 is not. Conversely, in position P2 illustrated in FIG. 6E, the second light emitting element 120 is emitting while the first light emitting element 118 is not. This enables the bi-colour LED array 406 to be toggled between a first colour associated with a first wavelength, for example, 660 nm, and a second colour associated with a second wavelength, for example, 830 nm.

A positive voltage is applied to terminal A with respect to terminal B when the switch is in position P1, and the first light emitting element 118 is illuminated whereas the second light emitting element 120 is illuminated when the polarity switch 440 is in position P2 by the negative voltage applied to terminal A with respect to terminal B.

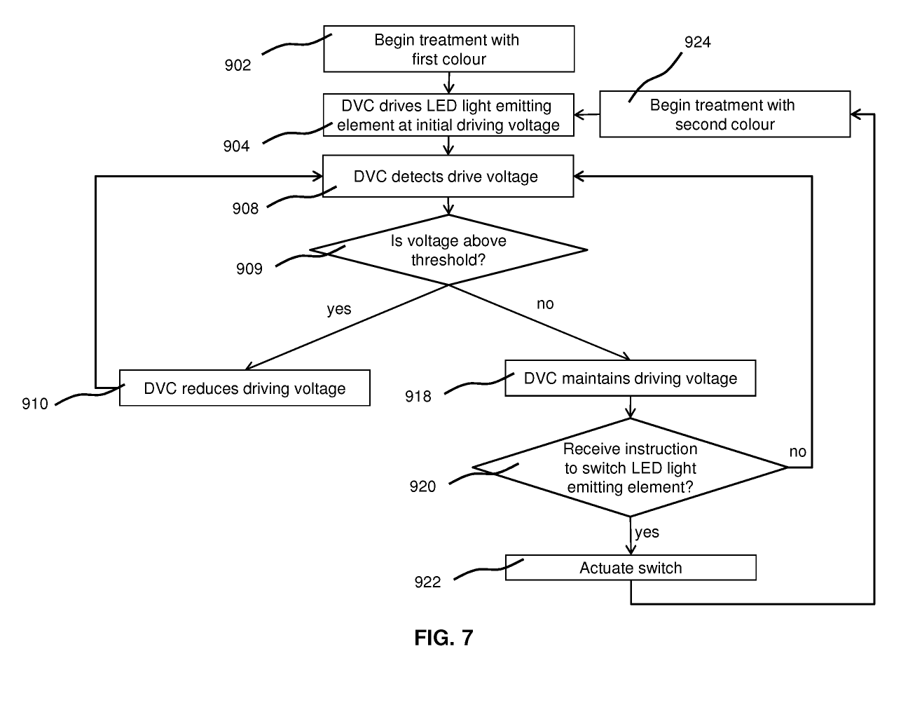

Referring to the process flow diagram of FIG. 7, an example polarity switching process is shown for alternating between colours of a bi-colour LED. At step 902, treatment is selected by the operation that initially requires operation of the first LED light emitting element 118, which emits at a first colour. At 904, the DVC 450 begins to drive the first LED light emitting element 118 at an initial driving voltage and the current controller 552 applies a predetermined current selected based on the treatment protocol being applied. At 908, the DVC 450 determines whether driving voltage is above the required LED array voltage.

At 909, the DVC 450 determines whether the drive voltage is above a selected voltage threshold corresponding to the required LED array voltage. The voltage threshold may be selected based on the type or specific model of light emitting element(s) being used. If the voltage is above the selected threshold at 909, the DVC 450 automatically reduces the driving voltage at 910. In this example, the voltage threshold is selected to be approximately 1 volt above the required LED array voltage. It will be appreciated that the DVC 450 may continuously monitor the voltage to determine whether the voltage has exceeded the selected threshold voltage (e.g. associated with the LED array).

The DVC 450 may set the voltage according to a pre-established mapping of the required voltage characteristics of the array of LED light emitting elements being driven.

Returning to 909, if the voltage is below the selected threshold (e.g. an upper threshold associated with the LED array), the DVC 450 maintains the voltage at 918. At 920, if the switch receives an instruction from controller 542 to switch the light emitting element, the polarity switch 440 is actuated at 922 and treatment is begun with a second colour at 924 and the DVC 450 drives the second LED light emitting element at an initial driving voltage. If the switch 440 does not receive an instruction to switch the light emitting element, the DVC 450 detects the drive voltage at 908.

The DVC 450 may drive the second LED light emitting element at the same initial driving voltage in 904 as the first LED light emitting element. Alternatively, each light emitting element may be provided with a separate initial driving voltage selected based on the electrical properties of the light emitting element.

The current controller 552 is operable to drive and set the driving current of, each of the arrays of the LED light emitting elements. As outlined below, the current applied to an LED light emitting element affects the intensity of light output by the light emitting element.

The controller 542 is operable to instruct the switch 440 to actuate based on an input from the user via the user interface 106 (e.g. communicatively coupled to the switch 440) or based on a selected treatment protocol. The controller 542 is also operable to instruct the switch 440 to actuate based on a treatment protocol being implemented by the treatment head control module 104.

It will be appreciated that LED light emitting element 118 and LED light emitting element 120 may be incorporated together as a bi-colour LED with the first LED light emitting element 118 representing the LEDs that emit colour when driven with a forward voltage and the second LED light emitting element 120 representing the LEDs that emit colour when driven with a reverse voltage. It will also be appreciated that each light emitting element 118 and 120 may comprise an array of LEDs.

The user interface 106 enables an operator to select entire treatment protocols from the treatment database 108 and load these onto the treatment head control module 104. The user interface 106 also provides the user with the capacity to customize these protocols, if necessary. The user interface 106 may also enable the operator to link a patient with a particular treatment protocol.

The display 110 of the user interface 106 can also be used to guide the operator through the steps of loading a treatment protocol from the treatment database 108, customizing the treatment protocol, and administering the treatment in accordance with the treatment protocol. For example, the user interface 106 may provide an operator with specific instructions for a treatment protocol including timing of when to move the treatment head, a diagram of the placement of a treatment head on the patient, etc.

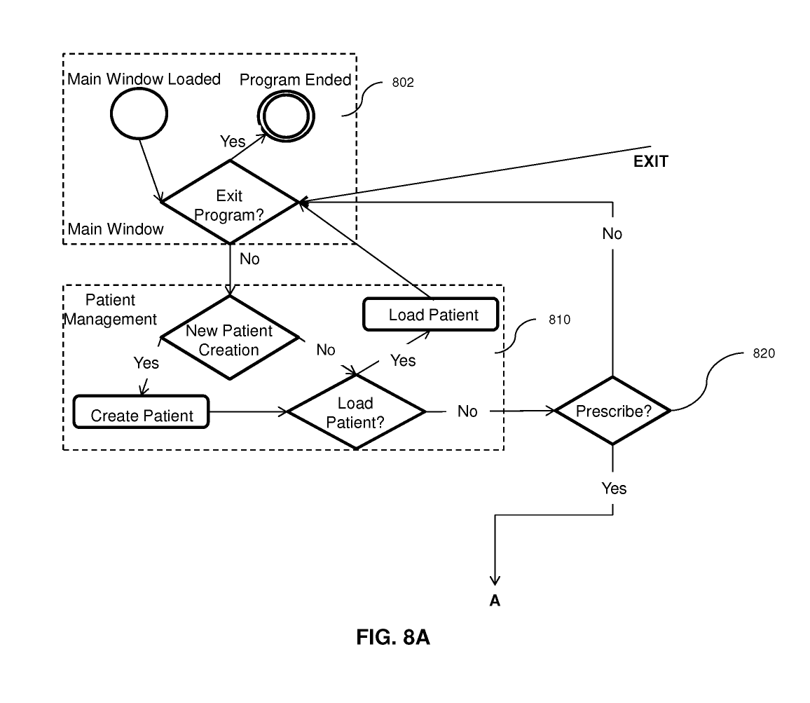

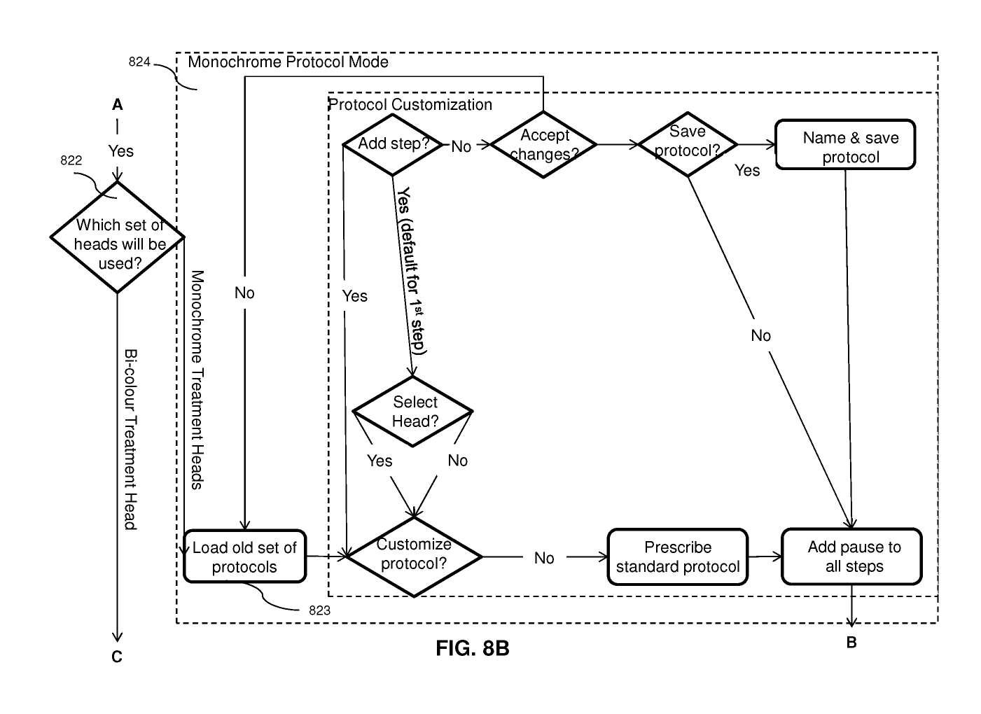

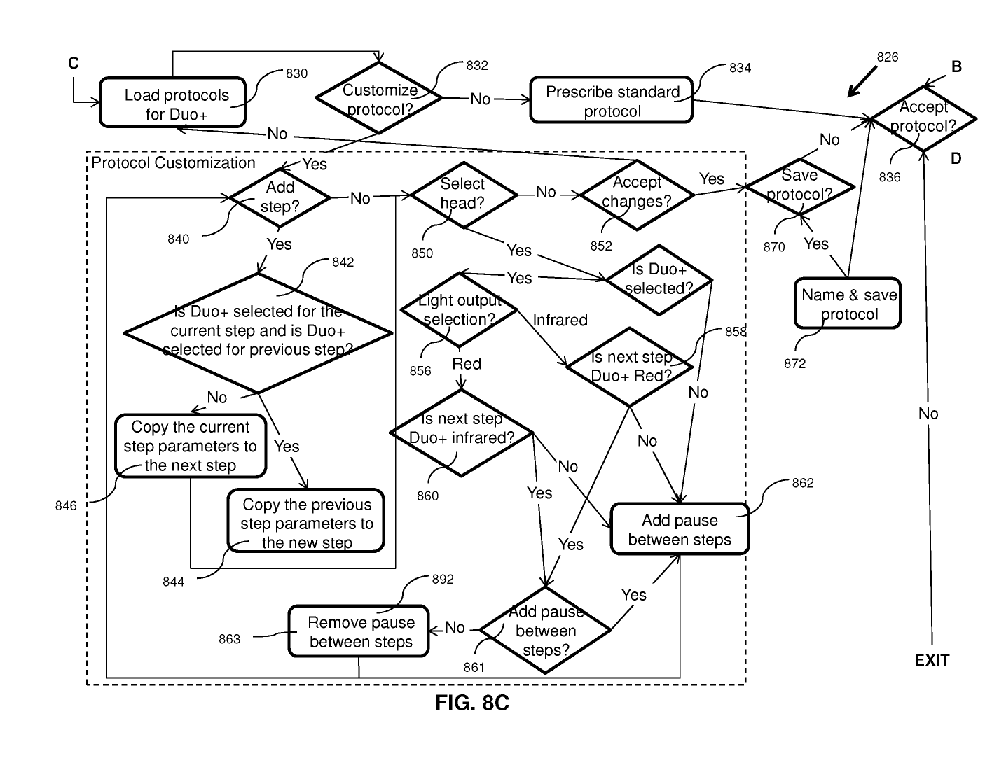

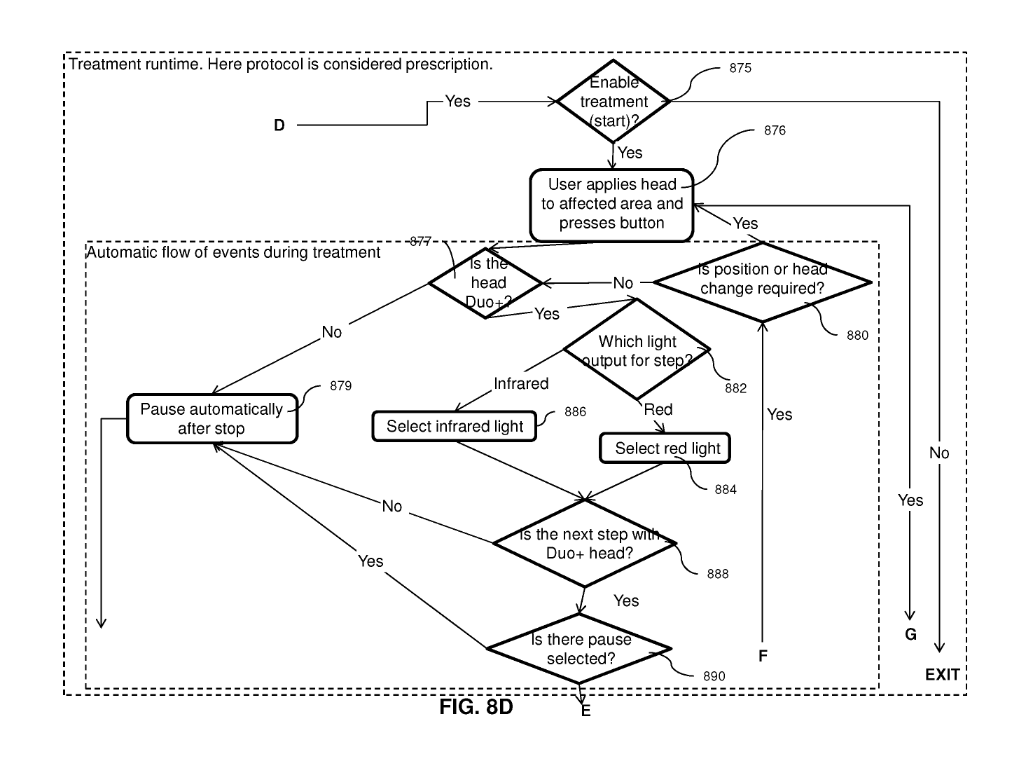

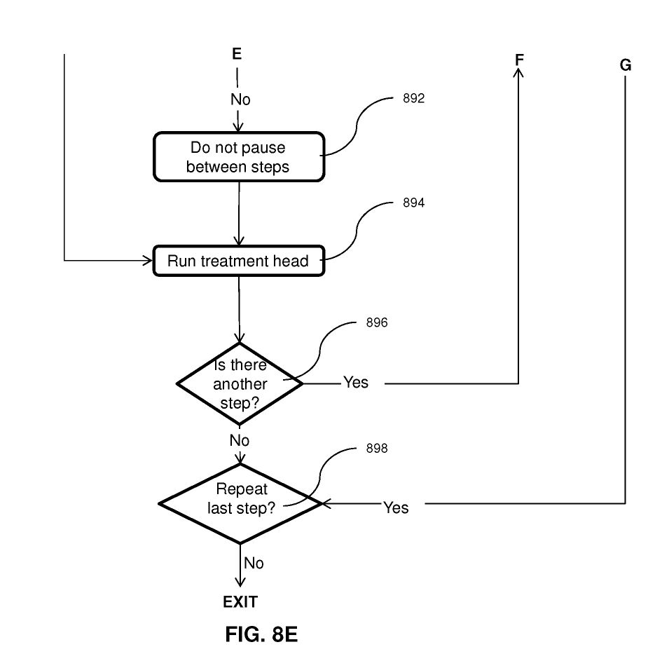

Referring now to FIG. 8A to 8E, a simplified flow diagram of an operator selecting or creating a treatment protocol for a light treatment system 100 is provided. In step 810, the user interface 106 initiates a patient management interface whereby the operator is queried whether a patient's profile should be loaded at 810. A patient's profile may comprise patient health information, treatment history, injury diagnosis, prescriptions, and other relevant information.

If the operator selects to load a patient's protocol via the user interface 106, the treatment head control module 104 loads the patient's protocol. This may involve the treatment head control module 104 selecting a treatment protocol from the treatment database 108 and loading the treatment protocol into its memory.

Should the operator select not to load a patient in 810, the interface queries the operator whether to prescribe a treatment at 820. If the operator elects not to prescribe a treatment at 820, the user interface 106 may query whether to exit the program. If the operator does not wish to exit the program, the operator may elect to create a new patient profile in 808, causing the user interface 106 to enable the operator to enter patient information in 806.

If the operator elects to prescribe a treatment in 820, the user interface 106 then queries whether a bi-colour light source will be used at 822. If a bi-colour light source is not to be used, a set of protocols for a traditional monochrome light source is loaded at 823 and the light treatment system 100 operates in the monochrome protocol mode. However, if the operator elects to use a bicolour light source at 822, the interface enables the operator to configure the light treatment system for a bi-colour light source at 826 and protocols for bi-colour LED sources are selected at 830.

At 832, the operator is provided with the option to customize a protocol. Turning back to step 832, if the operator elects not to customize the protocol in 832, the standard protocol is prescribed at 834 and can be accepted at 836. If the operator elects to customize a protocol, the user interface 106 queries whether to add a treatment step at 840. If the operator elects to add a treatment step at 840, the user interface queries whether a bi-colour treatment head is selected for both the new step and the previous step in 842. If the bi-colour treatment head was selected for both the current step and the previous step in 842, the previous step parameters are applied to the new step in 844. If the bi-colour treatment head was not selected for both the current step and the previous step, the current step parameters are selected to be used with the new step in 846.

If the operator elects not to add a treatment step at 840, the operator is asked whether to select the light treatment head 102 that will be used. If an operator elects to accept the changes to the protocol at 852, the operator is provided with the option to save these changes at 870. If the operator decides to save the changes, the operator is provided with an interface for entering the name of the protocol and the protocol is saved at 872. Whether or not the operator elects to save the protocol, the operator is given the option of accepting the protocol at 836 to move ahead to the treatment interface.

At 822, the user interface 106 queries the operator whether a bi-colour treatment head 102 is selected. If not, a pair of monochrome treatment heads must be used and the treatment head control module 104 includes a pause between each of the treatment steps. If a bi-colour treatment head is selected in 822, the user interface 106 queries which colour of light is to be output at 568. If a first wavelength is selected, for example, a red wavelength at 660 nm, the user interface 106 queries whether a second wavelength, for example, an infrared wavelength at 830 nm is selected at 860. Similarly, if the second wavelength is selected at 856, the user interface 106 queries whether to select the first wavelength at 858.

If, at 860, the operator inputs that the next step is another application at the first wavelength, the user interface 106 queries whether to add a pause between the current step and the next step at 861. If an operator elects to add the pause, a pause is added at 862.

If, at 860, the operator inputs that the next step is another application of the first wavelength, a pause is added automatically at 862. If, at 858, the operator inputs that the next step is another application of the second wavelength, a pause is not required at 862. Alternatively, if at 858, the operator inputs that the next step is not the alternate wavelength, the operator is queried whether to add a pause between the current step and the next step at 861. If an operator elects not to add a pause between the current step and the next step, no pause is added at 863.

A treatment comprising three steps is provided in tables 1, 2, and 3, below wherein CW refers to a continuous wave having substantially no modulating waveform. It will be appreciated that although position 1 and position 2 on the patient are shown with the same parameters, the frequency, duty cycle, exposure time, and other parameters may be varied depending on the location of treatment on a patient's body. For example, regions of a patient's body that are deeper within body tissues may require a longer treatment time, a higher intensity, and a longer wavelength for deeper penetration. LD-I 200 refers to a treatment head comprising an 840 nm light source.

In an example treatment protocol, the user applies a first wavelength from the treatment head 102 to a treatment area of a patient's body for a selected period of time. After the selected period of time, the user applies a second wavelength from the treatment head 102 to the same region of the patient's body for a second selected period of time. If another area is to be treated, the user may, after the second selected period of time, move the treatment head 102 to treat a different area of the patient's body and repeat the delivery of the first and second wavelengths. This process is repeated until the area of the patient that requires treatment has been treated. Treatment of a selected number of treatment areas, or all treatment areas, may be repeated. In an examples shown in tables 1 to 3 below, the first wavelength emitted by the treatment head 102 is a red wavelength and the second wavelength emitted by the treatment head 102 is an infrared wavelength.

After area that requires treatment has received a full treatment, a laser treatment may be applied. The laser treatment may comprise a high intensity treatment which is applied principally to the area of the patient which is the most affected by the condition being treated. The laser treatment may be a red laser treatment or an infrared laser treatment. For example, the laser treatment may comprise a 100 mW red laser treatment or a 200 mW infrared laser treatment. The duration of the laser treatment may be, for example, 3 to 5 minutes.

TABLE 1
Stage One

| | Frequency | Duty Cycle | Duration |
|---|---|---|---|
| Treatment Head | (Hz) | (%) | (minutes) |
| Position 1 | | | |
| Bi-colour + Red | CW | | 5 |
| Bi-colour + | 10→20 | 40→80 | 7 |
| Infrared | | | |
| Position 2 | | | |
| Bi-colour + Red | CW | | 5 |
| Bi-colour + | 10→20 | 40→80 | 7 |
| Infrared | | | |
| LD-I 200 | CW | | 6 |

TABLE 2
Stage Two

| | Frequency | Duty Cycle | Duration |
|---|---|---|---|
| Treatment Head | (Hz) | (%) | (minutes) |
| Position 1 | | | |
| Bi-colour + Red | CW | | 5 |
| Bi-colour + | 20→50 | 30→80 | 7 |
| Infrared | | | |
| Position 2 | | | |
| Bi-colour + Red | CW | | 5 |
| Bi-colour + | 20→50 | 30→80 | 7 |
| Infrared | | | |
| LD-I 200 | CW | | 6 |

TABLE 3
Stage 3

| | Frequency | Duty Cycle | Duration |
|---|---|---|---|
| Treatment Head | (Hz) | (%) | (minutes) |
| Position 1 | | | |
| Bi-colour + Red | 500→1000 | 70→90 | 5 |
| Bi-colour + | 250 | 80→90 | 7 |
| Infrared | | | |
| Position 2 | | | |
| Bi-colour + Red | 500→1000 | 70→90 | 5 |
| Bi-colour + | 250 | 80→90 | 7 |
| Infrared | | | |
| LD-I 200 | 100 or CW | 90 | 6 |

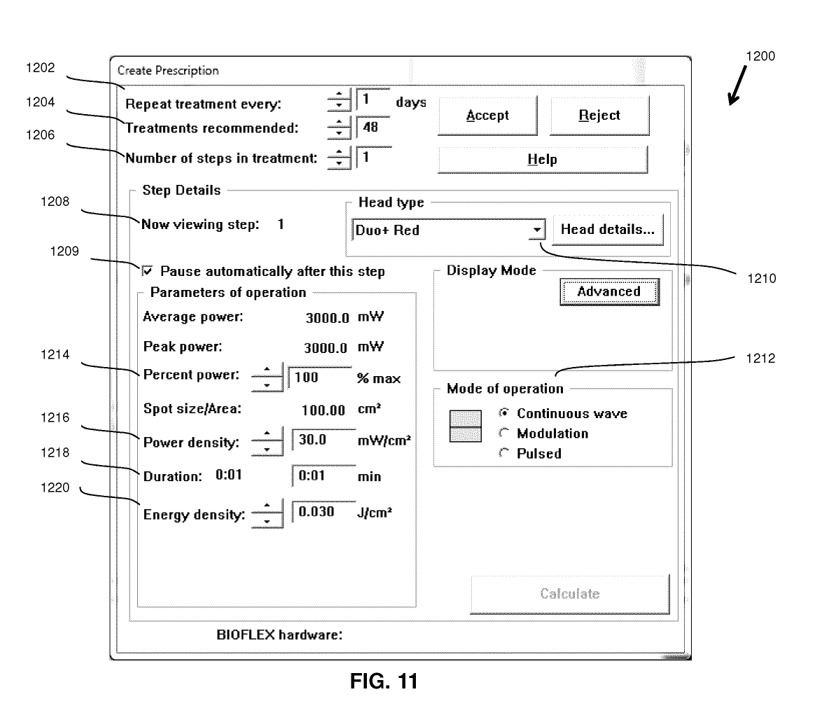

In each of Tables 1 through 3, the frequency is defined as the number of cycles per second that a driving waveform repeats. The duty cycle is the percentage of a single representative cycle in which a waveform is in the “on” condition. Power density and energy density, which are defined in mW per square centimeter and J per square centimetre, respectively, can be selected as outlined below with reference to FIG. 11. The power density is the flux of light from a light source which is incident on a predetermined area. Energy density is the total amount of radiant energy incident on a predetermined area and within a set period of time.

Various waveforms, in addition to continuous waves, may be applied in a modulated mode. For example, the light treatment system 100 may operate in a modulation mode using a substantially square waveform, a substantially sinusoidal waveform, or a substantially triangular waveform. The waveform can be selected to control the output intensity of the light source 103. For example, a square wave may simply switch the light source 103 between an “on” condition and an “off” condition. A sinusoidal or triangular wave may be used to modulate the intensity of the light source 103 by gradually alternative between a maximum in the “on” state and a minimum in the “off” state such that the intensity of the light brightens and dims in the “on” state according to the waveform.

Once the operator has elected to accept the protocol in 836, the operator is presented with the option to begin treatment of the patient at 875. If the operator elects not to begin treatment, the operator is returned to the main window of the user interface 802 and presented with the option to exit the program at 812. If the operator elects to proceed with treatment, the user interface 106 instructs the operator to apply the treatment head 102 to the area to be treated in 876 and activate the treatment head 102.

Once the operator activates the treatment head, the treatment head control module 104 determines whether the treatment head 102 is a bi-colour treatment head. If the treatment head 102 is determined to be a bi-colour treatment head, the treatment controller 104 determines the wavelength of light which is to be used for the current treatment step at 882. If the wavelength is determined to be the first wavelength, for example, a red wavelength, the light emitting element for the first wavelength is selected at 884. Alternatively, if the wavelength is determined to be the second wavelength, for example, an infrared wavelength, the light emitting element for the second wavelength is selected at 886.

Although FIG. 8D presents a selection between a first wavelength and a second wavelength, it will be appreciated that both wavelengths may be emitted by the treatment head 102 simultaneously.

Once the first step of treatment is complete, the treatment head control module 104 initiates the next treatment step using the bi-colour treatment head 102 at 888, which has been specified in the protocol and may display an indication to the operator on the user interface 106 regarding the next step of treatment. If a pause is selected in the treatment protocol after each step, a pause is introduced after the current step at 879 and the treatment head is run at 894 after the selected duration of the pause has elapsed.

If the treatment protocol does not contain a pause, a pause is not introduced at 892 and the treatment head is run immediately at 894.

If another step should be run after the current step at 896, and if a change in position of the treatment head on the patient's body is required at 880, the operator is provided with a notification to move the head, for example, by a diagram that shows the required position of the head, and a pause enables the operator to activate the head at 876 to begin treatment in the new area of the patient. If no position is required, the user interface returns to 877.

If there are no additional steps at 896, and the light treatment system 106 should not repeat the step of treatment that was completed most recently at 898, the user interface 106 returns to the main window 802.

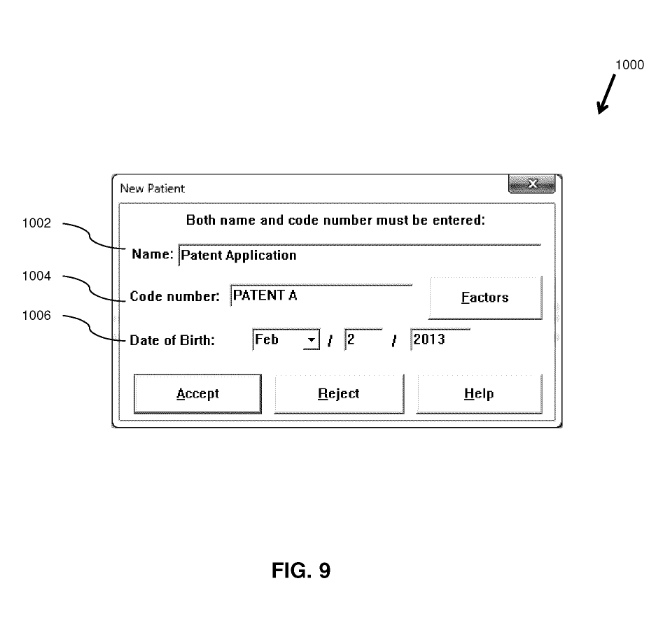

Referring now to FIG. 9, a patient information interface 1000 for entering and viewing patient information is provided. Although fields for entering patient name 1002, patient identifier 1004, and date of birth 1006 are shown, it will be appreciated that various other relevant patient information may be included. For example, treatment history, medical conditions, treatment plan, and other information may be provided to the patient information interface 1000.

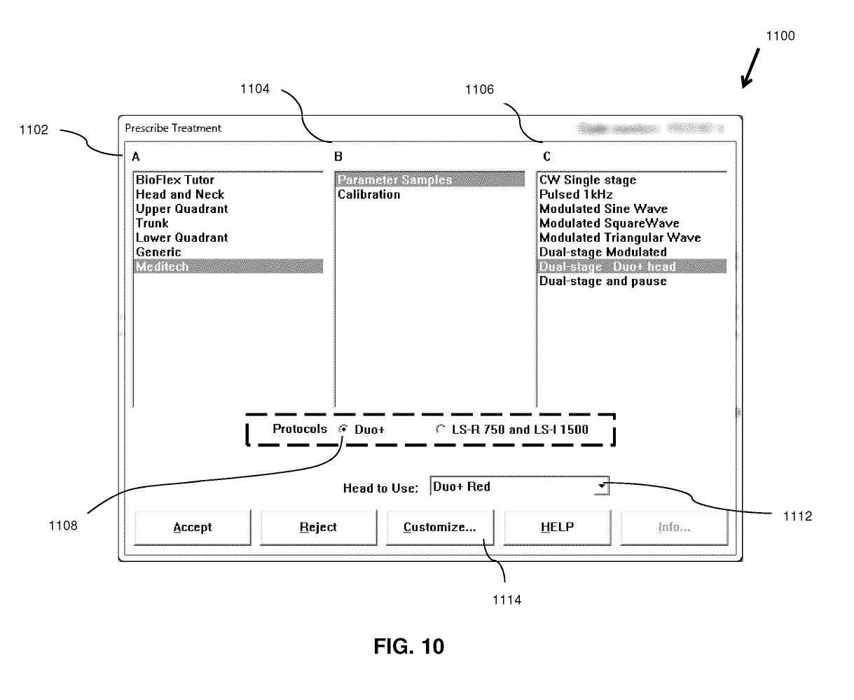

Treatment may be prescribed using a treatment prescription interface 1100, as shown in FIG. 10. The interface of FIG. 10 enables an operator to select among predefined treatment protocols. On the treatment prescription interface 1100, the operator may select whether a single colour treatment head or a multi-colour treatment head is used. The operator may also select an area to be treated, treatment parameters, the type of treatment protocols 1108, the treatment head 1110 being used, and the wave pattern of the applied light.

Treatment protocols may be selected based on various groupings. For example, column A 1102 may contain treatment protocols adapted for the body region of a patient. Upon making a selection in column A 1102, an operator is presented with a number of options in column B 1104. Column B 1104 may contain treatment protocols for a specific region of the patient's body or a specific ailment. Similarly, upon the operator making a selection from column B 1104, the operator may select and option from column C 1106, which may, for example, contain options to select among various intensities and/or waveforms. The operator is therefore able to select a characteristic from one or more of the columns to determine an appropriate treatment protocol. The operator may also be provided with an interface element 1114 to access an interface to enable the operator to prepare customized treatment protocols, as shown in FIG. 11.

FIG. 11 shows a screen capture of an example interface for entering customized treatment protocols. For example, fields may be provided for an operator may enter the frequency 4102, in number of days, at which a treatment is to be repeated, the total number of treatments that have been prescribed 4104, and the number of steps required in a treatment 4106. FIG. 11 also provides the operator with fields to adjust the power applied 1214 (e.g. by varying the intensity of light applied to the patient), the power density of the light 1216, the duration 1218, and, consequently, the energy density 1220 of a step of treatment.

The operator may further be able to select the mode of operation at 1212. Example modes of operation include a continuous wave, a modulated wave, or a pulsed signal. The operator is also given an indication of which step of the treatment the operator is presently viewing at 4108. The operator may select between various treatment heads at 1210.

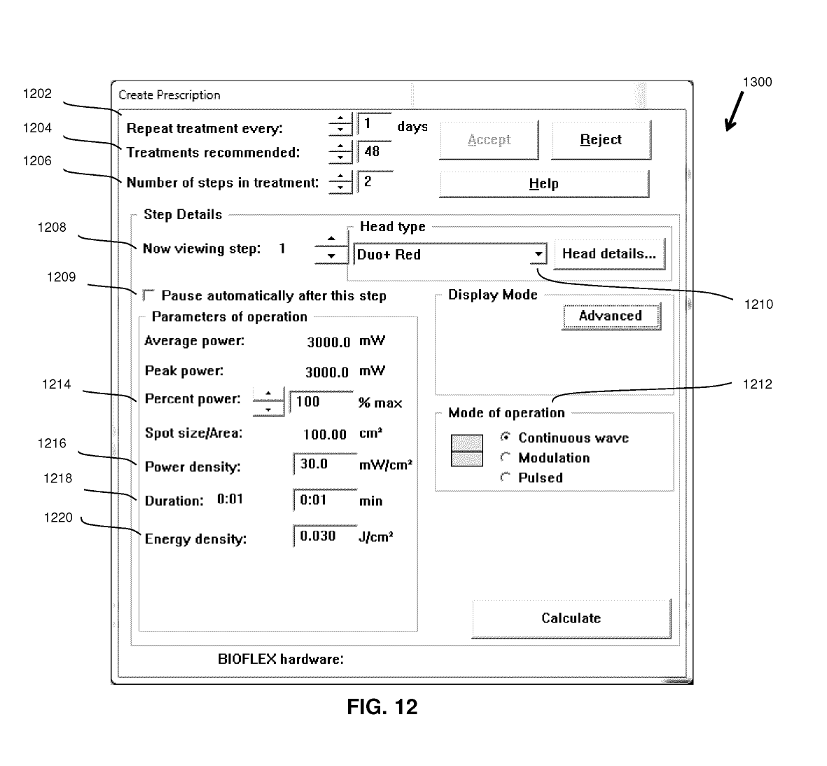

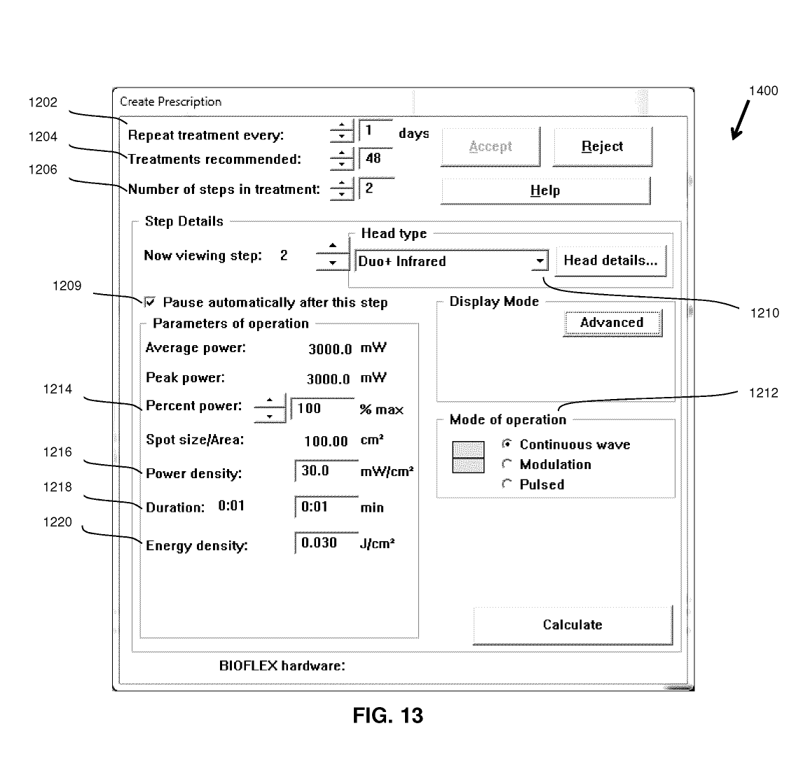

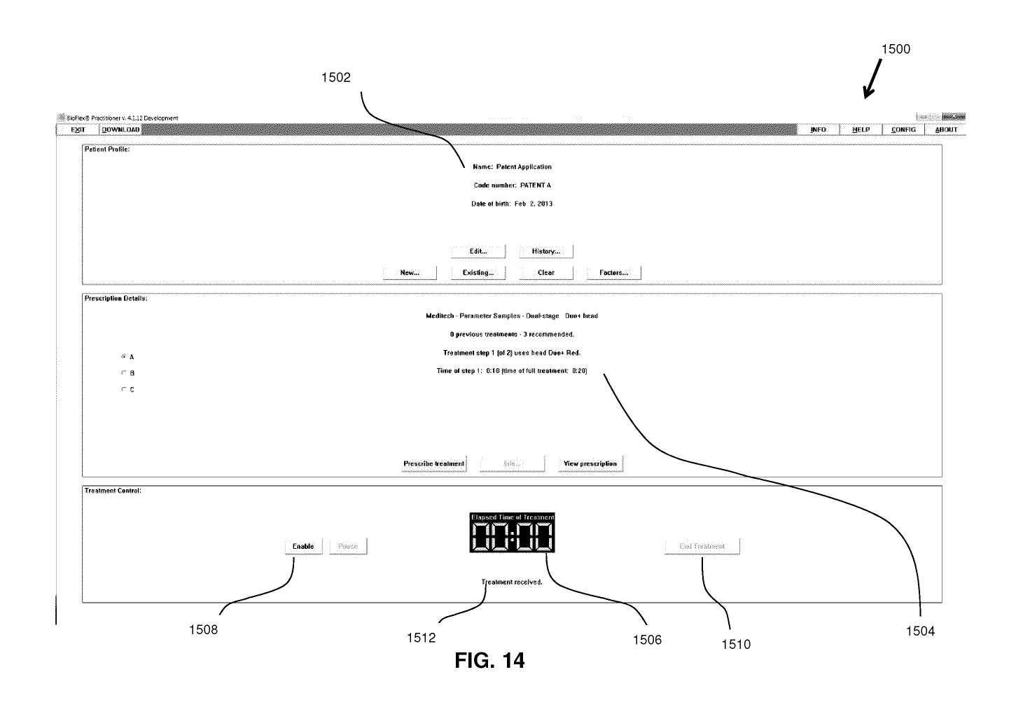

FIGS. 12 and 13 are example user interfaces for entering two steps of a treatment protocol implemented using a bi-colour treatment head. Referring to FIG. 12, a user interface 1300 similar to that of FIG. 11 is provided. FIG. 12 shows the parameters of the first of a two-step treatment in which a red wavelength, for example, 660 nm, is used. It will be appreciated that the parameters associated with the treatment head 102 may be selected for each step of each treatment. For example, the first step of a treatment may emit light at a power density of 30 mW per square centimetre whereas the second step of treatment may emit light at a power density of 22 mW per square centimetre. FIG. 13 shows the parameters of the second of the same two-step treatment in which an infrared wavelength, for example, 840 nm, is used.

While treatment is being administered to a patient, an information display 1500 may be provided to the operator, as is shown in FIG. 14. The information screen may display the patient's personal information, the patient's treatment history, and the number of treatments prescribed for the future in field 1502. The information display 1500 may further display information regarding an on-going treatment including time elapsed 1506, whether a treatment is complete 1512 time remaining for treatment, treatment head temperature, any warning indications, the time until the treatment head must be moved, or a diagram of the patient showing the current location of the treatment head and the next location of the treatment head at field 1504.

The information display 1500 may also provide an option for an operator to end an ongoing treatment at 1510. Upon the operator enabling the treatment at 1510, the operator may then initiate treatment with the treatment head 102, for example, by actuating a switch on the treatment head 102 or by simply applying the treatment head 102 to the skin of the patient.

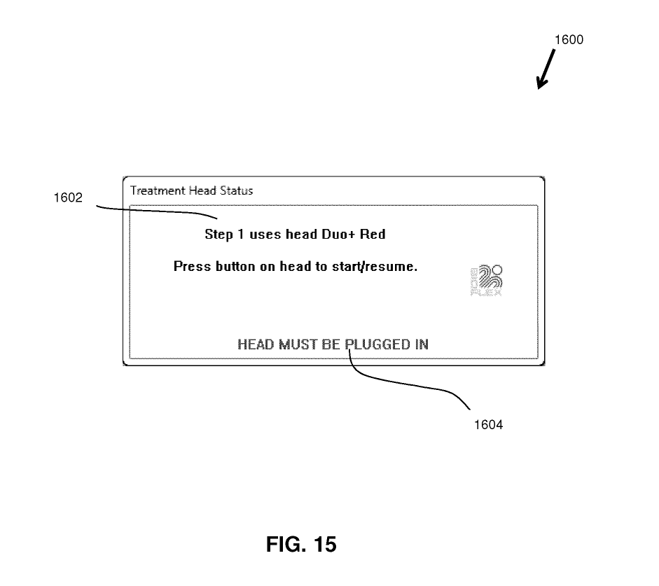

As is shown in FIG. 15, a treatment head status display 1600 may show the present status of the treatment head 1604 and provide an input for a user to initiate, pause, or stop treatment 1602. The status of the treatment head may include, for example, the treatment head being: ready for operation, in operation, under an error condition, overheated, or in an off condition. The status display 1600 may also provide an operator-selectable option to change the condition of the treatment head 102, for example, from an operating condition to an off condition or from a ready for operation condition to an operation condition.

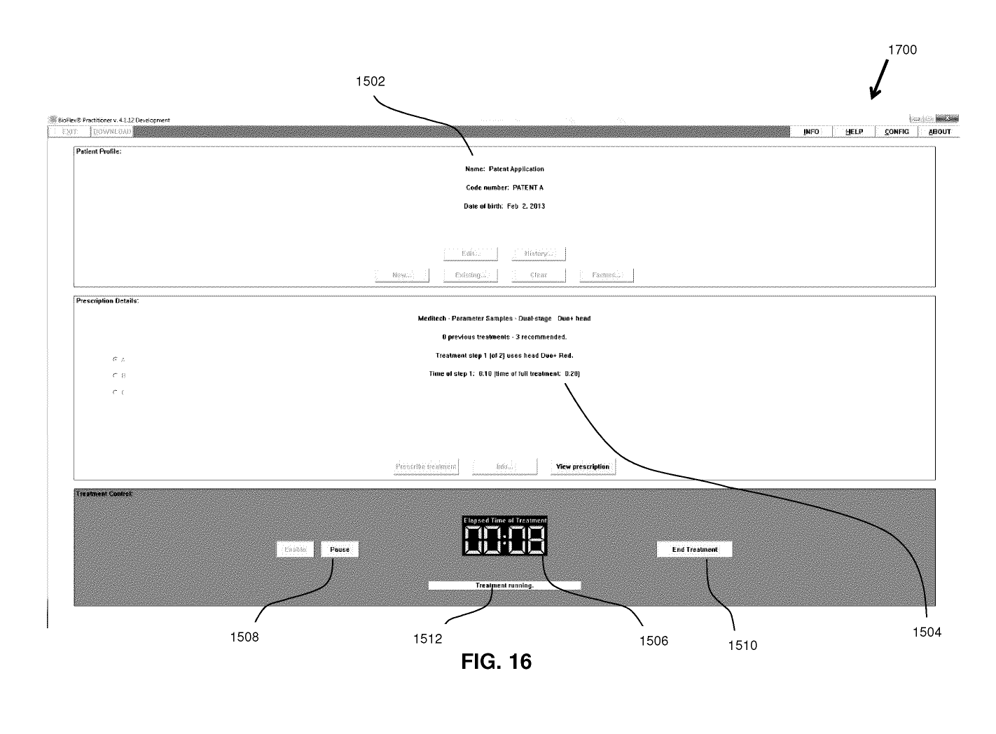

Referring to FIG. 16, an information display 1700 similar to that of FIG. 14 is provided. The information display shows the status of the treatment as on-going and indicates that 8 seconds have elapsed since the beginning of treatment.

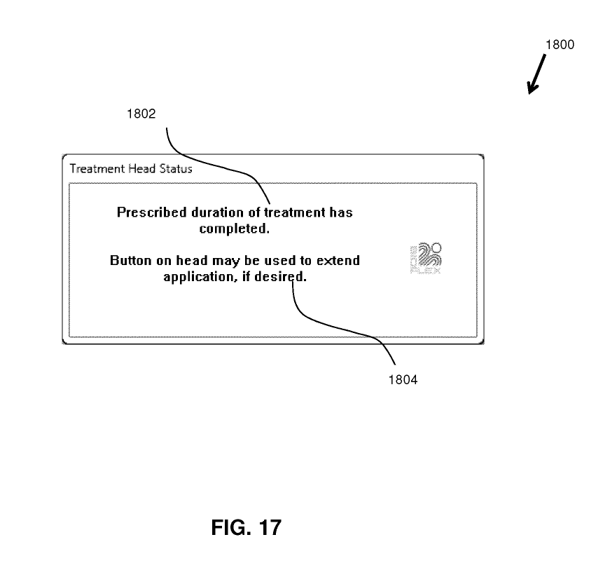

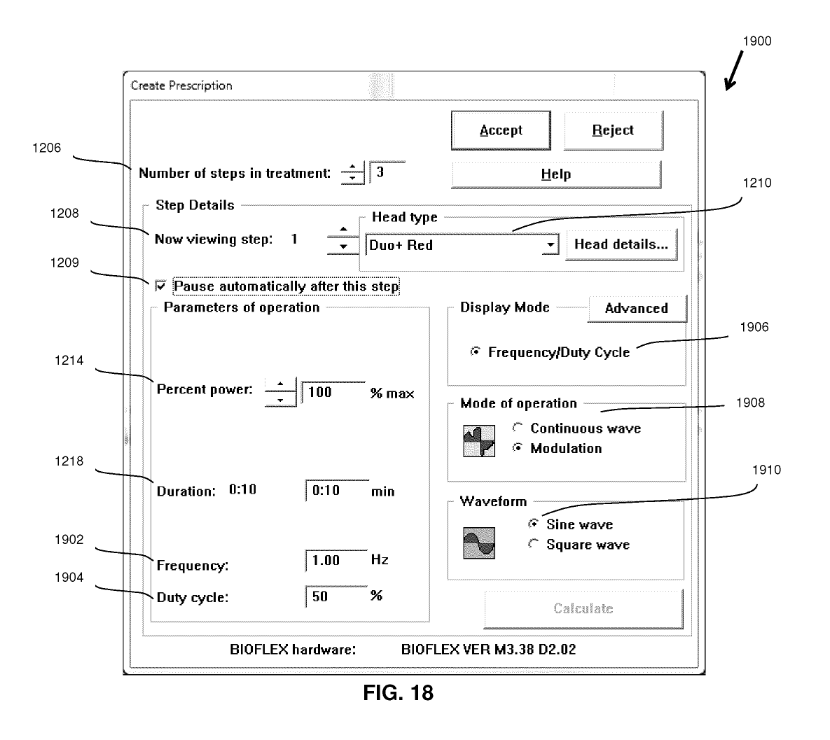

Turning to FIG. 17, a treatment head status display 1800 is provided indicating that the status of the treatment is complete 1802. Although the treatment is complete, an option is provided to extend the treatment upon receiving an input from the user 1804. As such, for an operator may provide additional treatment to the patient if the operator deems this additional treatment necessary, or if a portion of the initial treatment was not properly administered. Referring now to FIG. 18, a user interface 1900 similar to those of FIGS. 12 through 14 is provided, however, in the user interface 1900 shows the light treatment system 100 operating in a modulation mode 1908 with a sine wave 1910. As such, additional options enabling the operator to select the frequency of the sine wave 1902 and the duty cycle of the sine wave 1904 are provided. The operator may elect to display or hide fields for entering optional parameters at 1906, for example, by actuating a radio toggle button.

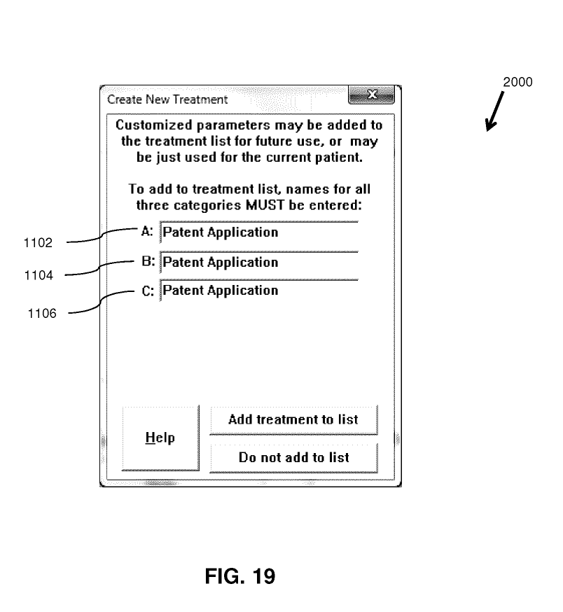

FIG. 19 enables an operator to store a customized treatment protocol in the treatment database according to the groupings outlined above with reference to FIG. 10 such that the treatment protocol may be accessed at a later time.

Although reference is made to emitting light at specific wavelengths, it will be appreciated that the spectral width of these wavelengths may vary. It will also be appreciated that emitting light at two or more wavelengths includes emitting light at as a substantially continuous spectrum, regardless of the relative intensity of any peaks present in the spectrum. In other words, the light source 103 may emit light at wavelengths other than the specific target wavelengths. It will also be appreciated that although reference is made to colours of light, the wavelengths may be within or outside of the visible spectrum, for example, the wavelengths may be infrared wavelengths, near-infrared wavelengths, or even UV wavelengths.

Moreover, the light source can be emitting white light that includes several wavelengths in the spectrum. By way of example, white light may include several wavelengths in the visible spectrum. The white light may, for example, include all colors in the spectrum. Specific wavelengths emitted by the light source may be emitted by using wavelength-selective filters. For example, 660 nm wavelength can be generated from a white light source (or any other light source comprising light at 660 nm) and a 660 nm selective filter which allows at least a significant proportion of 660 nm wavelength light to transmit while substantially blocking the rest of the spectrum. The selective filter can be made of various suitable materials and shapes including, but not limited to, flat lenses, convex or concave lenses or even fiber optics.

What is claimed is:

1. A system configured for multi-colour light treatment, the system comprising:
a treatment head comprising:
a first light emitting element operable to emit light at a first wavelength;
at least one other light emitting element operable to emit light at a second other wavelength; and,
a switch coupled to said treatment head, the switch configured for alternately switching between the first and other light emitting element in dependence upon pre-defined treatment criteria.

2. The system of claim 1, further comprising:
a treatment head control module communicatively coupled to the treatment head, the treatment head control module comprising instructions for defining said pre-defined treatment criteria, the treatment head control module for providing at least one of: an intensity of emitted light; a duration of light emission at a particular wavelength; number of cycles of treatment at each wavelength; and a selection of at least two wavelengths to the treatment head for controlling operation of the light emitting elements.

3. The system of claim 2, further comprising:
a treatment database coupled to the treatment head control module, the treatment database comprising pre-defined treatment protocols associated with at least one patient, the pre-defined treatment protocols for further defining said pre-defined treatment criteria.

4. The system of claim 2, further comprising a user interface communicatively coupled to the treatment head control module, the treatment head control module configured for receiving input from the user interface for further defining said pre-defined treatment criteria.

5. The system of claim 1, wherein the switch further comprises a polarity switch operable to toggle between a forward and a reverse polarity to cause the treatment head to alternately emit light at the first wavelength by the first light emitting element and at the second wavelength of the other light emitting element.

6. The system of claim 5, wherein the forward polarity is driven with a forward voltage and current and the reverse polarity is driven with a reverse voltage and current.

7. The system of claim 6, further comprising a current controller coupled to said first light emitting element, and said other light emitting element, the current controller for setting a pre-defined current value for driving a respective one of said first and said other light emitting element, the pre-defined current value associated with a characteristic of said light emitting element.

8. The system of claim 7 wherein the current value set by the current controller is further associated with a pre-defined light intensity associated with the emitted light.

9. The system of any one of claims 1 to 8, further comprising a dynamic voltage controller and wherein a selected one of the first and the other light emitting element is configured to emit light in dependence upon receiving, from said dynamic voltage controller, a forwardly biased input driving voltage above a pre-defined characteristic threshold voltage of the selected one light emitting element.

10. The system of claim 9, wherein the voltage controller automatically adjusts the driving voltage to cause a constant current to be driven through the selected light emitting element at a selected current value associated with the respective light emitting element.

11. The system of claim 9, wherein once the switch switches from the first to the other light emitting element, the voltage controller is configured to dynamically adjust the driving voltage to a pre-defined voltage value associated with the other light emitting element.

12. The system of claim 7, wherein the current controller is further configured to receive an input from at least one of a user interface and a pre-defined treatment protocol stored on a memory for instructing the switch to activate based on said input.

13. The system of claim 1, wherein the treatment head further comprises a temperature sensor for sensing a temperature associated with the treatment head and a current limiter configured for limiting electrical current flowing through said light emitting elements in dependence upon said sensed temperature being upon a pre-defined temperature value associated with the light emitting elements.

14. The system of claim 13, wherein the current limiter comprises a positive temperature coefficient thermistor placed in series connection with the light emitting elements.

15. The system of claim 1 further comprising a power control module comprising the switch, a current controller coupled to the switch and the light emitting elements for adjusting a current applied to said first and other light emitting elements, and a dynamic voltage controller for applying a driving voltage to each said first and other light emitting elements.

16. The system of claim 15 wherein the power control module is positioned on the treatment head and electrically coupled thereto.

17. A method for multi-colour light treatment, comprising:
applying a forward driving voltage to a bi-colour LED to cause the LED to emit at a first wavelength, the LED comprising a forward voltage upper threshold and a reverse voltage upper threshold;
orienting the bi-colour LED to emit onto a treatment area of a patient;
monitoring the voltage applied to the LED; and
upon the voltage exceeding the forward upper voltage threshold, reducing the driving voltage of the LED.

18. The method of claim 17, further comprising:
ceasing to apply a forward driving voltage to the bi-colour LED;
applying a reverse driving voltage to the bi-colour LED to cause the LED to emit at a second wavelength; and
upon the voltage exceeding the reverse upper voltage threshold, reducing the driving voltage of the LED.

19. The method of claim 17, wherein the first wavelength is substantially y in the infrared region of the electromagnetic spectrum.

20. The method of claim 18, wherein the second wavelength is substantially in the red region of the electromagnetic spectrum.

21. A light treatment device comprising:
a treatment head having:
a first LED light emitting element comprising an upper voltage threshold, the first light emitting element being operable to emit at a first wavelength; and
a second LED light emitting element comprising an upper voltage threshold the second light emitting element being operable to emit at a second wavelength;
a current controller operable to apply a selected driving current to one of the first or second light emitting elements; and
a dynamic voltage controller operable to:
apply a driving voltage to one of the first or second light emitting elements;
monitor the voltage applied to the first or second light emitting element; and
reduce the driving voltage applied to the first or second light emitting element upon detecting a driving voltage that exceeds a pre-defined upper threshold.

22. A method for multi-colour light treatment, comprising:
placing a light treatment head in a first location to treat a first treatment area;
conducting a local treatment step in the first area comprising:
treating a patient with a first wavelength in the first location; and
subsequently treating the patient with a second wavelength in the first location.

23. The method of claim 22, further comprising:
moving the light treatment head to a second location to treat a second treatment area; and
repeating the local treatment step in the second location.

24. The method of claim 23, further comprising conducting a laser treatment step by exposing the patient to a light generated from a laser light source.

25. The method of claim 22, wherein the first wavelength is a substantially infrared wavelength.

26. The method of claim 22, wherein the second wavelength is a red wavelength.

27. The method of claim 22, further comprising subsequently treating the patient with a plurality of other wavelengths in the first location using the treatment head.

28. The method of according to any one of claims 22 to 27 using the device of claim 21.
